SEQUENCE LISTING (17 sequences)

SEQ ID NO: 1 - DNA, 22 bp, Artificial
  Synthetic Probe for MMP-9 Gln279Arg SNP
ctctacacccgggacggcaatg

SEQ ID NO: 2 - DNA, 24 bp, Artificial
  Synthetic Probe for MMP-9 Gln279Arg SNP
actctacacccaggacggcaatgc

SEQ ID NO: 3 - DNA, 19 bp, Artificial
  Synthetic sense primer for MMP-9 Gln279Arg SNP
tctccccctttcccacatc

SEQ ID NO: 4 - DNA, 19 bp, Artificial
  Synthetic antisense primer for MMP-9 Gln279Arg SNP
tgtgctgtctccgccttct

SEQ ID NO: 5 - DNA, 10 bp, Artificial
  Synthetic Probe for MMP-9 Gln279Arg SNP
tctacacccg

SEQ ID NO: 6 - DNA, 10 bp, Artificial
  Synthetic Probe for MMP-9 Gln279Arg SNP
gggacggcaa

SEQ ID NO: 7 - DNA, 10 bp, Artificial
  Synthetic Probe for MMP-9 Gln279Arg SNP
tctacaccca

SEQ ID NO: 8 - DNA, 10 bp, Artificial
  Synthetic Probe for MMP-9 Gln279Arg SNP
aggacggcaa

SEQ ID NO: 9 - protein, 707 aa, Homo sapiens
  MISC_FEATURE (279)..(279): Xaa is Gln in the common variant and Arg in the rare variant
MSLWQPLVLVLLVLGCCFAAPRQRQSTLVLFPGDLRTNLTDRQLAEEYLYRYGYTRVAEMRGESKSLGPALLLLQKQLSLPETGELDSATLKAMRTPRCGVPDLGRFQTFEGDLKWHHHNITYWIQNYSEDLPRAVIDDAFARAFALWSAVTPLTFTRVYSRDADIVIQFGVAEHGDGYPFDGKDGLLAHAFPPGPGIQGDAHFDDDELWSLGKGVVVPTRFGNADGAACHFPFIFEGRSYSACTTDGRSDGLPWCSTTANYDTDDRFGFCPSERLYTXDGNADGKPCQFPFIFQGQSYSACTTDGRSDGYRWCATTANYDRDKLFGFCPTRADSTVMGGNSAGELCVFPFTFLGKEYSTCTSEGRGDGRLWCATTSNFDSDKKWGFCPDQGYSLFLVAAHEFGHALGLDHSSVPEALMYPMYRFTEGPPLHKDDVNGIRHLYGPRPEPEPRPPTTTTPQPTAPPTVCPTGPPTVHPSERPTAGPTGPPSAGPTGPPTAGPSTATTVPLSPVDDACNVNIFDAIAEIGNQLYLFKDGKYWRFSEGRGSRPQGPFLIADKWPALPRKLDSVFEEPLSKKLFFFSGRQVWVYTGASVLGPRRLDKLGLGADVAQVTGALRSGRGKMLLFSGRRLWRFDVKAQMVDPRSASEVDRMFPGVPLDTHDVFQYREKAYFCQDRFYWRVSSRSELNQVDQVGYVTYDILQCPED

SEQ ID NO: 10 - DNA, 7639 bp, Homo sapiens
  misc_feature (2665)..(2665): n is a in the common variant and g in the rare variant
tcaccatgagcctctggcagcccctggtcctggtgctcctggtgctgggctgctgctttgctgcccccagacagcgccagtccacccttgtgctcttccctggagacctgagaaccaatctcaccgacaggcagctggcagaggtgggcaaacacctagtctagagttggggagggctgtccgtgagggtgttgagtgtcccagagaggatgcagggcctcagaggagatgctttaggggtgtgttggtggtgatgggcgtatctgaagaacagaggtgtccagggttaggcagtggggggtcttgtggaggctttgagcagtgatggccagaaatgggcaatggggctttcctaggtgggaaatgggaaatggtttggggtgggggaggcattggagggttctggggtaagcataggctgggagtgaacaggggcaaaccttatgcagctgtggggtagaaatgggctagaggcatccaggggtgagaaggagctgaggatgtctaaggaggggagatccctgggtggtcagaaagcactggtgtctggaaagcatttaatgctttattaaatgttagtccctgctgggcatgacggctcacacttgtaatcccagcactttgggaggctgaggtggtaggatcgctgaagctcaggagtttgagcccagcctaggcaacatagtaagatcctgtctctacaaaaaaattaaagaaatagccaggcacagtgatgtgcacctgtagttccagctatgcagaaggctgagatgggaggatcgcttgagtccaggaggtccaggctgcagtgggctgataccgtctctccgaaaaagaaaaagaaaaaagactccctccatgagtgtctggagggagtcctttggccccagctgggcagagaaaggggtcagagatctggcatgtgtgtgtcccttcatccacaggaatacctgtaccgctatggttacactcgggtggcagagatgcgtggagagtcgaaatctctggggcctgcgctgctgcttctccagaagcaactgtccctgcccgagaccggtgagctggatagcgccacgctgaaggccatgcgaaccccacggtgcggggtcccagacctgggcagattccaaacctttgagggcgacctcaagtggcaccaccacaacatcacctattggtgagccggggccgtgggggcagcggggtggggcggggaggccaggtctggctcttgggccagcggtgaacatgtcctgtcttggacgcgtccctgggtttcactatttaatgtgtggcccctggggagtgtccccacctctgagcctctgtttctccttcagggaaatggctcttgcaatccaagtcctcctgccagggccattgtgagggtctaagtagacaaaaaaaaaaaaaaaaaaaacagtctggaagcaatttatagatgagagcgtggacggcagagagcattgtgtatgttgaagtctctgcgatatggggtgtccctgctgccccgctccagcctttcacttctgacctccttcctctggctcttacgctacaggatccaaaactactcggaagacttgccgcgggcggtgattgacgacgcctttgcccgcgccttcgcactgtggagcgcggtgacgccgctcaccttcactcgcgtgtacagccgggacgcagacatcgtcatccagtttggtgtcgcgggtgagaacgtgaggagggaaaatccaagagacctgggcggggtcagggaagggaggaccacggagagcgtggaggcagcagtggccccggcttcctcttgcctgcccgcgctgccctggcttatacggcccctcctgccagacagtgcacagggccagggcgccaggctgggagagcttcgcgcaggcgggatttcagcccgcacttatttcggagcccttgccttgggcagcgcacaatctgcgcagcagtactcggctaaccctcttcctctcgacctgtttcttcagagcacggagacgggtatcccttcgacgggaaggacgggctcctggcacacgcctttcctcctggccccggcattcagggagacgcccatttcgacgatgacgagttgtggtccctgggcaagggcgtcggtgagattctgagtcctcctggcccctgattcccttcattctctcccactcatcacccgccgccctaactccggtcccccctcctcctgcagtggttccaactcggtttggaaacgcagatggcgcggcctgccacttccccttcatcttcgagggccgctcctactctgcctgcaccaccgacggtcgctccgacggcttgccctggtgcagtaccacggccaactacgacaccgacgaccggtttggcttctgccccagcgagagtgagtgagggggctcgccgagggctgggggcgcccaccacccttgatggtcctgggttctaattccagctctgccactagtgctgtgtggcctgcaattcaccctcccgcactctgggcccaattttctcatctgagaaatgatgagagatgggatgaactgcagaccatccatgggtcaaagaacaggacacacttgggggttataatgtgctgtctccgccttctccccctttcccacatcctcctcgccccaggactctacacccnggacggcaatgctgatgggaaaccctgccagtttccattcatcttccaaggccaatcctactccgcctgcaccacggacggtcgctccgacggctaccgctggtgcgccaccaccgccaactacgaccgggacaagctcttcggcttctgcccgacccgaggtacctccaccctgtctaccaggttcagccccgccctctcatcatgtattggcccccaaaacgcggctcttccctcccatcagtttgtctttccactctcattggtcctcaggacgaccgtgactccgcccacctacaccacatttccaccactatccctgacttccaatggccccgccccagccactaaggttcggccttttctgcccagctggccgcctcttccttggtctggtgtcccaggcaccgcccacgggtctagcctcttctcaggagtgctctacagcgccccctaggccaccaagattgtttagctccctgtcgggtcggcccctgactccttattggactcatccatctggctcatccaaggccttgggtctctccagctgactcgacggtgatggggggcaactcggcgggggagctgtgcgtcttccccttcactttcctgggtaaggagtactcgacctgtaccagcgagggccgcggagatgggcgcctctggtgcgctaccacctcgaactttgacagcgacaagaagtggggcttctgcccggaccaaggtaggcgtggtcccgcggctccggggctggggttcccggcagtggtggtggtggggtggccagggctgggggctcggcccggcgctcacgtctcaggctccctctccctccaggatacagtttgttcctcgtggcggcgcatgagttcggccacgcgctgggcttagatcattcctcagtgccggaggcgctcatgtaccctatgtaccgcttcactgaggggccccccttgcataaggacgacgtgaatggcatccggcacctctatggtgaggcaggggcagggatgggaggaggaggggaaagggcgtggctgtgccacagtaccaaagaattgggggttggggatcgggggaggaacggggcgtgcaggagaggtgggacctcaacgtctgtctggaagcagagcctgggcccagtcgctgccatgtcagtgcttagaggtggtgataaagagactctagagagagataggtgtgacttcaaaagccagtctactctgggcatggtggctcacgcctctaatcccagggctttgggagacccaaggcgggaggattgcttaagcccaggagttccagaccagcctcggcaacatagccagactcccatctctacaaaaaataaatgagcaaggcgtgaaggcacatgtctgtagtcctagctactctggaggctgaggtgggaggatctcttgagcccaggagttcgaggctgtagtgagctatgattgcaccactgcattccatcctgggccatagaggatgtcgcttaaaacgaaaaagaagaagaagaaagtcctgtggtttgggaagggaggctgagtgaggaggggcctgtgtgccagaggaggcttcactgagaagcttaggggagcagatgttctaggggtacagaggtatgcaggaataggaagagtctcaccccgtgtctctttttaggtcctcgccctgaacctgagccacggcctccaaccaccaccacaccgcagcccacggctcccccgacggtctgccccaccggaccccccactgtccacccctcagagcgccccacagctggccccacaggtcccccctcagctggccccacaggtccccccactgctggcccttctacggccactactgtgcctttgagtccggtggacgatgcctgcaacgtgaacatcttcgacgccatcgcggagattgggaaccagctgtatttgttcaaggatgggtgaggaggcggggttgtgtggatgcgggagggggctttgcggaggggctgcccgtcccttcccgcccactggccctgtgtccaaggcttagagcccgtcctttccctcctcgctttctcaggaagtactggcgattctctgagggcagggggagccggccgcagggccccttccttatcgccgacaagtggcccgcgctgccccgcaagctggactcggtctttgaggagcggctctccaagaagcttttcttcttctctggttagttacctactttccctcccccgcccggtcaatccccatcagtcaaggaggctcaagagaccatcgataacccacgaaacgtcttgtgcgttttagaaaaatacgccccctggcggacgcagtttagcaaacgtaggggcggctgagtttctgccccctcctctccacgccctcgcgtcgctctacccagcgcctctgcccctgggttgcagggactgcgggcacgcgggctaggaaaggcctcgccggaatctccctcctcgcgttctaggagtacgtgctccctctgcgcccccaaaccgacgtgaccctcctcccctgcagggcgccaggtgtgggtgtacacaggcgcgtcggtgctgggcccgaggcgtctggacaagctgggcctgggagccgacgtggcccaggtgaccggggccctccggagtggcagggggaagatgctgctgttcagcgggcggcgcctctggaggtgagcgccgccgcggccgccggcagggggagcccgggcgccgtcggtccgtccgctagccggctcagcacctgtctcctccgcgcctgcccgcaggttcgacgtgaaggcgcagatggtggatccccggagcgccagcgaggtggaccggatgttccccggggtgcctttggacacgcacgacgtcttccagtaccgaggtgagggctgaggaggatcccttcgtgagacaccacactaagctcctcttagtgagtggtcaaattctgagcgaggaagaaaaagcccttggaaatggaaacaaatgccccagcacagacaagatcccagcagaggcagaggccttctccaggtcatttaggaagtcagggatgcaaccaagaccaggacccagatttcctgcctccccggctggaagctctttctccttcagtacaggacggcaggtggtttgtatggaagctcagttattagacaacagtcatcaagtgccgataatgtgccaggcactgtgctacagggagagataagacaattcacagctctgtgactttgggcaagtcactgcttctctactcttcgagcctcagtttccccatctgtaatatggggactatagctggaattacacttgacttccctttcttaccagtcacatccaaacagttgacaaggtgaacaagatttcctgccaccaaaatctttttcgagtctgtcattttttttgccatcttctttataaacaccccagcccaaaccatactggctgtccaggacctttaacaaattccatgagattagagagggggtaggagtgaagggcaatggtcttgggagtgaccccagatgaattccaaggtcaaagaaattaagaggatctgacactccacccccgtgttctcatctcttcccactcctcctgttatttactctgctccacccacactggctgctctttgaacagatcaaggtcattcctagcttacagcctttgtgccagttgttccctctgtctggaaagcttcccctccagattgtcactgggccatcccactgtcttccttcaggtttcagtgctaaggccattgcttcaatgaggccttctttgatgcttattatctatttacttgtttttattttctccatagctttctatattttctttttttttcttttttctttttttttttttttgagatggagtcttgctctgtcgcccaggctggagtgcagtggcacgatctcagctcactgcaacctccgcctcccgggttcaagcgattctcctgcctcagcctcccaagtagctgggattacaggtgcctgccaccacgcttggctaattttttgtattttttagtagagacggggtttcaccatcttggccaggctggtcttgaactcctgacctcgtgatccacccgcctcagcctcccaaagtgctgggattacaggcatgagccaccgcacccagccgctttctatattttcaaaaccaatctcatttatttatgtgtttgcttaattgtctcttgcctcactagagtgtaagcaccaagataattgagatcatgcctgcattttttctgcttatccccagtatcttgaacaaagcacatagtagatgctcagtaaatgatgaatgaacagatttgttcaatgaatgagcgttgaatgaattgttctgagcattaagatagttggtctattcatttgttaattcattcacaaaatgtgtatggtgtacctactgtgtgctaggctctgtggcagtgctttgggcactgaggtctgtgccctccagcatctcacagaacctcacagcatctcacaggttggggggatggaggtgatatgtgaaaaccttagaaagttctagaaatggcagaagagatggttgtcaagatcttgttcctatttctgtatatgtgggagaattagaatcactcctcttatgcctgcctgtctcctgcagagaaagcctatttctgccaggaccgcttctactggcgcgtgagttcccggagtgagttgaaccaggtggaccaagtgggctacgtgacctatgacatcctgcagtgccctgaggactagggctcccgtcctgctttggcagtgccatgtaaatccccactgggaccaaccctggggaaggagccagtttgccggatacaaactggtattctgttctggaggaaagggaggagtggaggtgggctgggccctctcttctcacctttgttttttgttggagtgtttctaataaacttggattctctaaccttt

SEQ ID NO: 11 - DNA, 2373 bp, Homo sapiens
  misc_feature (841)..(841): n is a in the common variant and g in the rare variant
tcaccatgagcctctggcagcccctggtcctggtgctcctggtgctgggctgctgctttgctgcccccagacagcgccagtccacccttgtgctcttccctggagacctgagaaccaatctcaccgacaggcagctggcagaggaatacctgtaccgctatggttacactcgggtggcagagatgcgtggagagtcgaaatctctggggcctgcgctgctgcttctccagaagcaactgtccctgcccgagaccggtgagctggatagcgccacgctgaaggccatgcgaaccccacggtgcggggtcccagacctgggcagattccaaacctttgagggcgacctcaagtggcaccaccacaacatcacctattggatccaaaactactcggaagacttgccgcgggcggtgattgacgacgcctttgcccgcgccttcgcactgtggagcgcggtgacgccgctcaccttcactcgcgtgtacagccgggacgcagacatcgtcatccagtttggtgtcgcggagcacggagacgggtatcccttcgacgggaaggacgggctcctggcacacgcctttcctcctggccccggcattcagggagacgcccatttcgacgatgacgagttgtggtccctgggcaagggcgtcgtggttccaactcggtttggaaacgcagatggcgcggcctgccacttccccttcatcttcgagggccgctcctactctgcctgcaccaccgacggtcgctccgacggcttgccctggtgcagtaccacggccaactacgacaccgacgaccggtttggcttctgccccagcgagagactctacacccnggacggcaatgctgatgggaaaccctgccagtttccattcatcttccaaggccaatcctactccgcctgcaccacggacggtcgctccgacggctaccgctggtgcgccaccaccgccaactacgaccgggacaagctcttcggcttctgcccgacccgagctgactcgacggtgatggggggcaactcggcgggggagctgtgcgtcttccccttcactttcctgggtaaggagtactcgacctgtaccagcgagggccgcggagatgggcgcctctggtgcgctaccacctcgaactttgacagcgacaagaagtggggcttctgcccggaccaaggatacagtttgttcctcgtggcggcgcatgagttcggccacgcgctgggcttagatcattcctcagtgccggaggcgctcatgtaccctatgtaccgcttcactgaggggccccccttgcataaggacgacgtgaatggcatccggcacctctatggtcctcgccctgaacctgagccacggcctccaaccaccaccacaccgcagcccacggctcccccgacggtctgccccaccggaccccccactgtccacccctcagagcgccccacagctggccccacaggtcccccctcagctggccccacaggtccccccactgctggcccttctacggccactactgtgcctttgagtccggtggacgatgcctgcaacgtgaacatcttcgacgccatcgcggagattgggaaccagctgtatttgttcaaggatgggaagtactggcgattctctgagggcagggggagccggccgcagggccccttccttatcgccgacaagtggcccgcgctgccccgcaagctggactcggtctttgaggagccgctctccaagaagcttttcttcttctctgggcgccaggtgtgggtgtacacaggcgcgtcggtgctgggcccgaggcgtctggacaagctgggcctgggagccgacgtggcccaggtgaccggggccctccggagtggcagggggaagatgctgctgttcagcgggcggcgcctctggaggttcgacgtgaaggcgcagatggtggatccccggagcgccagcgaggtggaccggatgttccccggggtgcctttggacacgcacgacgtcttccagtaccgagagaaagcctatttctgccaggaccgcttctactggcgcgtgagttcccggagtgagttgaaccaggtggaccaagtgggctacgtgacctatgacatcctgcagtgccctgaggactagggctcccgtcctgctttggcagtgccatgtaaatccccactgggaccaaccctggggaaggagccagtttgccggatacaaactggtattctgttctggaggaaagggaggagtggaggtgggctgggccctctcttctcacctttgttttttgttggagtgtttctaataaacttggattctctaacctttaaaaaaaaaaaaaaaaaaaaaaaaaaaaaaaaaaaaaaaaaaaaaaaaaaaa

SEQ ID NO: 12 - DNA, 22 bp, Artificial
  Synthetic primer for CA repeat in MMP-9
aagccctttctcatgctggtgc

SEQ ID NO: 13 - DNA, 22 bp, Artificial
  Synthetic primer for CA repeat in MMP-9
tgacaggcaagtgctgactcag

SEQ ID NO: 14 - DNA, 20 bp, Artificial
  Synthetic sense primer for C/T-1562 polymorphism
gcctggcacatagtaggccc

SEQ ID NO: 15 - DNA, 20 bp, Artificial
  Synthetic antisense primer for C/T-1562 polymorphism
cttcctagccagccggcatc

SEQ ID NO: 16 - DNA, 31 bp, Artificial
  Primer for site directed mutagenesis
gagagactctacacccaggacggcaatgctg

SEQ ID NO: 17 - DNA, 30 bp, Artificial
  Primer for site directed mutagenesis
cagcattgccgtcctgggtgagagtctctc

(19) United States
(12) Patent Application Publication
(10) Pub. No.: US 2007/0037156 A1
(43) Pub. Date: Feb. 15, 2007

(54) Metalloproteinase gene polymorphism in copd

(75) Inventors: Yohannes Tesfaigzi, Albuquerque, NM (US); Steven A. Belinsky, Albuquerque, NM (US)

Correspondence Address: PEACOCK MYERS, P.C., 201 THIRD STREET, N.W., SUITE 1340, ALBUQUERQUE, NM, 87102, US

(73) Assignee: Lovelace Respiratory Research Institute, Alburquerque, NM (US)

(21) Appl. No.: 10/567,876

(22) PCT Filed: Aug. 11, 2004
(86) PCT No.: PCT/US04/26035

§ 371 Date: Feb. 8, 2006

Related U.S. Application Data
(60) Provisional application No. 60/494,631, filed on Aug. 11, 2003.

(51) Int. Cl. C12Q 1/68

(52) U.S. Cl. 435/6

(57) ABSTRACT

A method for genetic testing relating to chronic obstructive pulmonary disease (COPD), including testing for a single nucleotide polymorphism within the metal loproteinase-9 gene, an exon 6, codon 279 Gln/Arg polymorphism, where in presence of the variant polymorphism, particularly homozygous presence, is predictive of susceptibility to COPD. Further provided are methods of selecting patients for specific therapies and methods of drug development and discovery for treatment and prevention of COPD.

### CROSS-REFERENCE TO RELATED APPLICATION

This application claims the benefit of the filing of U.S. Provisional Patent Application Ser. No. 60/494,631, entitled “Metalloproteinase Gene Polymorphisms In COPD”, filed on Aug. 11, 2003, and the specification thereof is incorporated herein by reference.

### BACKGROUND OF THE INVENTION

1. Field of the Invention (Technical Field)

The invention provides methods for the diagnosis, treatment, and/or prevention of various diseases and/or disorders, particularly chronic obstructive pulmonary disease (COPD) disorders, by determining the presence of a Gln279Arg polymorphism in the matrix metalloproteinase-9 (MMP-9) gene or expressed protein, polynucleotides, primers, and probes useful for such determination, and methods for drug development and discovery.

2. Description of Related Art

Note that the following discussion refers to a number of publications by author(s) and year of publication, and that due to recent publication dates certain publications are not to be considered as prior art vis-à-vis the present invention. Discussion of such publications herein is given for more complete background and is not to be construed as an admission that such publications are prior art for patentability determination purposes.

COPD constitutes a large and growing health problem; it is expected to be the fourth leading cause of death worldwide in 2020 (Murray, C. J., and A. D. Lopez. 1997. Alternative projections of mortality and disability by cause 1990-2020: Global Burden of Disease Study. Lancet 349 (9064): 1498-1504). COPD accounts for 13% of hospital admissions in the United States, and evidence suggests that its incidence is rising, particularly in women. Given the large increase in smoking in many foreign countries, COPD will become a larger worldwide problem in ensuing years (MacKenzie, T. D., et al. 1994. The human costs of tobacco use. N Engl J Med 330(14):975-980). Despite the enormous burden of the disease in health, economic, and personal perspectives (Feenstra, T. L., et al. 2001. The impact of aging and smoking on the future burden of chronic obstructive pulmonary disease. Am J Respir Crit Care Med 164:590-596), the underlying molecular mechanisms have not been elucidated, and treatment is focused on smoking cessation and treatment of symptoms.

COPD is a general term that includes several overlapping lung conditions that share the common functional problem of airflow limitation. Chronic bronchitis, one of these lung conditions, is defined clinically as chronic productive cough. Emphysema, which is defined pathologically, is determined functionally by the decrease in elastic recoil and the increased resistance in the airways. Asthma with fixed airflow obstruction is included in the American Thoracic Society (ATS) definition of COPD and may be characterized as obstruction with a large reversible component although there is no clear recommendation of the degree of reversibility.

COPD is associated with an accelerated decline in FEV₁ (forced expiratory volume in 1 second), reduced expiratory airflow, and airtrapping. It appears that COPD manifests through a chronic inflammatory state; however, it is not clear how this inflammation relates to the rapid decline in FEV₁.

The majority of COPD patients (90%) have regularly smoked cigarettes; therefore, environmental factors are clearly important in this disease (Silverman, E. K., and F. E. Speizer. 1996. Risk factors for the development of chronic obstructive pulmonary disease. Med Clin North Am 80(3):501-522). However, only 20-35% of chronic heavy smokers develop symptomatic COPD (Fletcher, C., and R. Peto. 1977. The natural history of chronic airflow obstruction. Br Med J 1 (6077):1645-1648) with some patients developing airflow obstruction at an early age (younger than age 40). This suggests that genetic factors are likely to be critical in determining which cigarette smokers are at risk of developing airflow obstruction.

COPD is a complex disease that is still poorly understood at the molecular level. A complex interplay between genetic and environmental factors is likely, and many distinct genes or groups of genes will be involved. In multifactorial diseases such as COPD, disease expression is influenced by interactions between multiple major and minor genes, and modulated by interacting non-genetic factors such as the environment or infection (Shapiro, S. D. 2001. End-stage chronic obstructive pulmonary disease: the cigarette is burned out but inflammation rages on. Am J Respir Crit Care Med 164:339-400). Despite the clinical importance of COPD, relatively few studies have searched for genetic factors using modern molecular genetic techniques.

Destruction of interstitial lung elastin is central to the degradation of alveolar septa and the pathogenesis of emphysema. Elastin degradation results in enlarged air spaces, less surface area for gas exchange, and loss of elasticity. Lung elastin is a long-lived connective tissue and once destroyed by elastolytic enzymes, emphysema will develop even though elastin may reaccumulate (Shapiro, S. D., et al. 1991. Marked longevity of human lung parenchymal elastic fibers deduced from prevalence of D-aspartate and nuclear weapons-related radiocarbon. J Clin Invest 87(5): 1828-1834).

Different cell types generate different classes of proteinases with differing modes of gene regulations. Neutrophils are short-lived and package active serine proteases in primary granules and MMP-9 in specific granules. Specific granule components are released in response to a variety of stimuli. Neutrophil activation leads to release of the primary granules (Liou, T. G., and E. J. Campbell. 1996. Quantum proteolysis resulting from release of single granules by human neutrophils: a novel, nonoxidative mechanism of extracellular proteolytic activity. J Immunol 157(6):2624-2631).

Monocyte precursors synthesize neutrophil elastase and cathepsin G in peroxidase-positive granules. Circulating monocytes can synthesize significant amounts of MMP-7 but either none or small amounts of MMP-1 and MMP-9. Alveolar macrophages can produce several MMPs including MMP-12 (macrophage elastase), MMP-1, MMP-9, smaller amounts of MMP-3, and MMP-7. Unlike neutrophils and monocytes, which store proteases potentially for rapid release, macrophages monitor and respond to their environment. Dysregulated expression of MMPs either directly or indirectly by cigarette smoke exposure could lead to the lung destruction characteristic of emphysema. Macrophages can also produce elastolytic cysteine proteases including cathepsins K, L, and S. If these proteinases are secreted in an acidic environment, they can cause significant lung destruction (Shapiro, S. D. 1999. The macrophage in chronic obstructive pulmonary disease. Am J Respir Crit Care Med 160(5 Pt 2):S29-S32).

While altered proteinase:antiproteinase balance may partially explain why only 15-30% of cigarette smokers develop clinically significant emphysema, susceptibility might also rest on the capacity for matrix repair after predictable proteolytic injury. It is likely that defective production of antiproteases may be important in the development of COPD, as is known for α₁-AT (alpha-1-antitrypsin), the major antiprotease that neutralizes neutrophil elastase activity. TIMPs (tissue inhibitors of metalloproteinases) are endogenous inhibitors of MMPs, and four TIMPs have now been characterized. Neutrophil elastase degrades TIMPs, potentiating protease activity.

Patients with low levels of α₁-AT develop early emphysema, particularly if they smoke. This was the first observation to associate a genetic defect with COPD (Ganrot, P. O., et al. 1967. Obstructive lung disease and trypsin inhibitors in alpha-1-antitrypsin deficiency. Scand J Clin Lab Invest 19(3):205-208). Since then, over 75 variants of α₁-AT have now been identified (Mahadeva, R., and D. A. Lomas. 1998. Genetics and respiratory disease. 2. Alpha 1-antitrypsin deficiency, cirrhosis and emphysema. Thorax 53(6):501-505). Patients homozygous for the Z variant of this enzyme (³⁴²Glu→Lys) have levels of α₁-AT that are only about 10% of normal and develop severe emphysema. The Z variant is only seen in Northern European populations and is rare in Asian and black populations. The S (²⁶⁴Glu→Val) and other variants of α₁-AT are not associated with COPD. A polymorphism in the 3′-promoter region of α₁-AT gene was found in 17% of patients with COPD compared with only 5% in the general population (Kalsheker, N. A., and K. Morgan. 1994. Regulation of the alpha 1-antitrypsin gene and a disease-associated mutation in a related enhancer sequence [published erratum appears in Am J Respir Crit Care Med 1995 March;151(3 Pt 1):926]. Am J Respir Crit Care Med 150(6 Pt 2):S183-S189). Another rare polymorphism in the 3′-promoter region has been associated with early onset COPD and was reported in three of 70 patients with COPD, but in none of 52 controls (Buraczynska, M., et al. 1987. Alpha 1-antitrypsin gene polymorphism related to respiratory system disease. Klin Wochenschr 65(12):538-541).

The genetic defect found in α₁-AT promoted the search for genetic abnormalities of other proteases. Two mutations were found in the α1-antichymotrypsin gene in a German population associated with COPD. One variant (²²⁷Pro→Ala) was found in four of 100 patients with COPD and the other (⁵⁵Leu→Pro) in three of 200 patients with COPD, while neither variant was found in any of the controls (Poller, W., et al. 1992. Mis-sense mutation of alpha 1-antichymotrypsin gene associated with chronic lung disease [letter]. Lancet 339(8808):1538); Poller, W., et al. 1993. A leucine-to-proline substitution causes a defective alpha 1-antichymotrypsin allele associated with familial obstructive lung disease. Genomics 17(3):740-743).

Although polymorphisms in a large number of genes have been determined, the polymorphisms of MMP genes that encode for central enzymes Involved in the progression of COPD have not been analyzed extensively in association with this disease. A role for the MMPs has been suggested in the pathogenesis of pulmonary emphysema (Barnes, P. J. 2000. Chronic obstructive pulmonary disease. N Engl J Med 343(4):269-80). Alveolar macrophages obtained from smokers have been reported to release more MMP-9 than those from nonsmokers (Lim, S., et al. 2000. Balance of matrix metalloprotease-9 and tissue inhibitor of metalloprotease-1 from alveolar macrophages in cigarette smokers. Regulation by interleukin-10. Am J Respir Crit Care Med 162 (4 Pt 1):1355-60), and increased expression of MMP-9 was observed in emphysematous samples compared to normal lung tissue (Ohnishi, K., et al. 1998. Matrix metalloproteinase-mediated extracellular matrix protein degradation in human pulmonary emphysema. Lab Invest 78(9): 1077-1087; Segura-Valdez, L., et al. 2000. Upregulation of gelatinases A and B, collagenases 1 and 2, and increased parenchymal cell death in COPD. Chest 117(3):684-694). Therefore, dysregulation of MMP-9 function may increase susceptibility for smoking-induced airway obstruction.

Minematsu et al. reported a significant increase in allelic frequency of the MMP-9 C-1562T promoter polymorphism in 45 smokers with emphysema compared with 65 matched smokers without emphysema as defined by high-resolution chest computed tomography (Minematsu, N., et al. 2001. Genetic polymorphism in matrix metalloproteinase-9 and pulmonary emphysema. Biochem Biophys Res Commun 289:116-119). A study compared smokers with COPD that showed a fast (n=284) or a slow (n=306) 5-year rate of decline in lung function (Joos, L., et al. 2002. The role of matrix metalloproteinase polymorphisms in the rate of decline in lung function. Hum Mol Genet 11(5):569-76). The variation in CA repeats in the promoter region of MMP-9 was not associated with the rate of decline in lung function, but MMP-1 G-1607GG and haplotypes consisting of alleles from the MMP-1 G-1607GG and MMP-12 Asn357Ser polymorphisms did show an association.

WO 99/57315 (filed as PCT/GB99/01446) discloses a number of genetic variants of MMP-9 gene, including the Gln279Arg polymorphism (called therein the exon 6 +23 guanine adenine variant). This application claims that Arg is the common variant. Further, this application does not disclose any association of this SNP with COPD or any related disease, and does not suggest or teach determination of this SNP for diagnosis, prognosis or therapy of COPD. U.S. Pat. No. 6,670,464 discloses a large number of SNPs identified for transcribed human sequences, including an SNP in MMP-7. However the MMP-7 SNP is apparently silent and non-coding, and does not appear to be associated with any disease or disorder.

### BRIEF SUMMARY OF THE INVENTION

In one embodiment the invention provides a method for determining the susceptibility of an individual to a chronic obstructive pulmonary disorder (COPD), which method includes the step of determining the presence of an exon 6 codon 279 Gln/Arg single nucleotide polymorphism within the matrix metalloproteinase-9 (MMP-9) locus in a biological sample obtained from the individual, wherein the 279 arginine polymorphism indicates susceptibility to chronic obstructive pulmonary disorder. In the method, an isolated nucleic acid molecule, which may be single-stranded or double-stranded DNA, cDNA or mRNA, can be used to detect the codon 279 Gln/Arg single nucleotide polymorphism. Preferably the isolated nucleic acid molecule is a probe which hybridizes under stringent conditions to a particular allele of the polymorphism, including the sequence 5′-CTCTACACCCGGGACGGCAATG (SEQ ID NO:1) or the sequence 5′-ACTCTACACCCAGGACGGCAATGC (SEQ ID NO:2). A nucleotide primer which amplifies a particular allele of the polymorphism may also be employed, which may be a 5′-TCTCCCCCTTTCCCACATC (SEQ ID NO:3) sense primer or a 5′-TGTGCTGTCTCCGCCTTCT (SEQ ID NO:4) antisense primer. Alternatively, the method can include determining the presence of an exon 6 codon 279 Gln/Arg single nucleotide polymorphism within the MMP-9 locus by testing expressed protein for the presence or absence of arginine in the 279 position.

In another embodiment, the invention provides a method of determining the efficacy of a substance to inhibit the 279Arg MMP-9 enzyme for use as a therapeutic or preventive agent for COPD, wherein the 279Arg MMP-9 enzyme is provided and the substance is tested for inhibition of the 279Arg MMP-9 enzyme. The step of providing the 279Arg MMP-9 enzyme can include inserting a gene expressing the 279Arg MMP-9 enzyme into a cell line, including SEQ ID NO:11 where 841 n is guanine (G). The method can also include providing the 279Gln MMP-9 enzyme, testing the substance for inhibition of the 279Gln MMP-9 enzyme, and comparing the results obtained for inhibition of the 279Arg MMP-9 enzyme with results obtained for inhibition of the 279Gln MMP-9 enzyme. In the method, either or both the 279Arg MMP-9 enzyme and the 279Gln MMP-9 enzyme may be purified enzyme.

In another embodiment, the invention provides a method of determining the efficacy of a substance to inhibit a 279Arg MMP-9 enzyme without substantially inhibiting a 279Gln MMP-9 enzyme for use as a therapeutic or preventive agent for COPD, wherein the 279Arg MMP-9 enzyme is provided, the substance is tested for inhibition of the 279Arg MMP-9 enzyme, the 279Gln MMP-9 enzyme is provided, the substance is tested for inhibition of the 279Gln MMP-9 enzyme, the results obtained for inhibition of the 279Arg MMP-9 enzyme are compared with results obtained for inhibition of the 279Gln MMP-9 enzyme, and the substance which inhibits the 279Arg MMP-9 enzyme without substantially inhibiting the 279Gln MMP-9 enzyme is selected. In this method, providing the 279Arg MMP-9 enzyme and the 279Gln MMP-9 enzyme can include inserting a gene expressing the 279Arg MMP-9 enzyme into a first cell line and inserting a gene expressing the 279Gln MMP-9 enzyme into a second cell line.

The invention further provides a method of treating a patient with COPD or at risk for developing COPD, wherein the presence of an exon 6 codon 279 Gln/Arg single nucleotide polymorphism within the MMP-9 locus is determined and an MMP-9 inhibitor administered to patients with the 279 arginine polymorphism. In the method, the MMP-9 inhibitor can be a selective 279Arg MMP-9 enzyme inhibitor.

A primary object of the present invention is to provide a method for determining susceptibility to COPD by determining the presence of an exon 6 single nucleotide codon 279 Gln/Arg polymorphism within the MMP-9 locus, wherein the 279 arginine polymorphism indicates susceptibility to chronic obstructive pulmonary disorder.

Another object of the present invention is to provide a method for use as an adjunct in a smoking cessation program.

Another object of the present invention is to provide a method for selecting patients for palliative or therapeutic COPD treatment by testing for the presence of an exon 6 single nucleotide codon 279 Gln/Arg polymorphism within the MMP-9 locus.

Another object of the present invention is to provide a method for developing and selecting an MMP-9 inhibitor for use in treatment of COPD.

Yet another object of the present invention is to provide a method for testing for the presence of an exon 6 single nucleotide codon 279 Gln/Arg polymorphism within the MMP-9 locus, by means of either a gene-based or protein-based assay.

Yet another object of the present invention is to provide variant and reference allele-specific oligonucleotides that hybridize to a nucleic acid molecule comprising a single nucleotide polymorphism (SNP) or to the complement of the nucleic acid molecule, which oligonucleotides can be probes or primers.

The invention thus provides oligonucleotides that can be used to amplify across a single nucleotide polymorphic site of the present invention. The invention further provides polynucleotides that may be used to sequence said amplified sequence. The invention further provides a method of analyzing a nucleic acid from a DNA sample using said amplification and sequencing primers to assess whether said sample contains the reference or variant base (allele) at the polymorphic site, comprising the steps of amplifying a sequence using appropriate PCR primers for amplifying across a polymorphic site, sequencing the resulting amplified product using appropriate sequencing primers to sequence said product, and determining whether the variant or reference base is present at the polymorphic site. The invention further provides a method of analyzing a nucleic acid from DNA samples from various ethnic populations using said amplification and sequencing primers to assess whether said samples contain the reference or variant base (allele) at the polymorphic site in an effort to identify populations at risk of developing COPD, comprising the steps of amplifying a sequence using appropriate PCR primers for amplifying across a polymorphic site, sequencing the resulting amplified product using appropriate sequencing primers to sequence said product, and determining whether the variant or reference base is present at the polymorphic site.

The invention further provides oligonucleotides that may be used to genotype DNA samples to assess whether said samples contain the reference or variant base (allele) at the polymorphic site. The invention provides a method of using oligonucleotides that may be used to genotype DNA samples to assess whether said samples contain the reference or variant base (allele) at the polymorphic site.

The invention provides a method of using oligonucleotides that may be used to genotype DNA samples to identify individuals that may be at risk of developing COPD or a COPD-related disease. The invention further provides a method of using oligonucleotides that may be used to genotype DNA samples to identify ethnic populations that may be at risk of developing COPD.

The invention further provides a method of analyzing a nucleic acid from an individual to determine genetic susceptibility to COPD. The method allows the determination of whether the reference or the variant base is present at the exon 6 single nucleotide codon 279 Gln/Arg polymorphism. This type of analysis can be performed on a number of individuals, who may also be tested (previously, concurrently or subsequently) for the presence of a disease phenotype. Thus the invention further relates to a method of predicting the presence, absence, or likelihood of the presence or absence, of a particular phenotype or disorder related to COPD and associated with a particular genotype. The method comprises obtaining a nucleic acid sample from an individual and determining the identity of one or more bases (nucleotides) at a specific (e.g., polymorphic) site of nucleic acid molecules described herein, wherein the presence of a particular base at the site is correlated with a specified phenotype or disorder, thereby predicting the presence, absence, or likelihood of the presence or absence of the phenotype or disorder in the individual, wherein the phenotype or disorder is preferably COPD or a COPD-related disorder.

The invention further relates to polynucleotides having one or more variant alleles. The invention further relates to polynucleotides of the present invention containing one or more variant alleles wherein said polynucleotides encode a polypeptide of the present invention. The invention relates to polypeptides of the present invention containing one or more variant amino acids encoded by one or more variant alleles.

The present invention also relates to antisense oligonucleotides corresponding to the polynucleotides of the present invention. Preferably such oligonucleotides are capable of discriminating against the reference or variant alleles of the polypeptide, preferably at one or more polymorphic sites of said polynucleotide.

The present invention also relates to antibodies, including antibody fragments, directed against polypeptides of the present invention. Preferably, such antibodies are capable of discriminating against the reference or variant alleles of the polypeptide at the polymorphic site of said polynucleotide.

The present invention also relates to recombinant vectors, which include the isolated nucleic acid molecules of the present invention, and to host cells containing the recombinant vectors, as well as to methods of making such vectors and host cells, in addition to their use in the production of polypeptides or peptides provided herein using recombinant techniques. Synthetic methods for producing the polypeptides and polynucleotides of the present invention are provided. Also provided are diagnostic methods for diagnosing diseases, disorders, and/or conditions related to the polynucleotides and polypeptides provided herein, and therapeutic methods for treating such diseases, disorders, and/or conditions. The invention further relates to screening methods for identifying binding partners of the polypeptides.

Thus the invention relates to a SNP in MMP-9, the codon 279 Gln/Arg SNP. The SNP may be detected as set forth herein, or by other means known in the art. It is further included and contemplated that the foregoing SNP may be used in combination with one or more other SNPs, including without limitation one or more of the following SNPs: C/T-1218, T/A-1702, C/T-861, C/T 138Ala/Val, and G/A 233Gln/Lys.

A primary advantage of the present invention is that it provides a genetic test for the susceptibility of a patient to COPD.

Another advantage of the present invention is that it provides a method of determining a population for whom preventive therapy, including smoking cessation programs and therapeutic drug therapies, are appropriate and most likely to be beneficial.

Yet another advantage of the present invention is that it provides a method for developing drugs and therapeutic substances for treatment or prevention of COPD and related disorders, including development of an inhibitor for MMP-9 enzyme activity, including the rare or 279 Arg form of MMP-9 enzyme activity.

Other objects, advantages and novel features, and further scope of applicability of the present invention will be set forth in part in the detailed description to follow, taken in conjunction with the accompanying drawings, and in part will become apparent to those skilled in the art upon examination of the following, or may be learned by practice of the invention. The objects and advantages of the invention may be realized and attained by means of the instrumentalities and combinations particularly pointed out in the appended claims.

### BRIEF DESCRIPTION OF THE SEVERAL VIEWS OF THE DRAWINGS

The accompanying drawings, which are incorporated into and form a part of the specification, illustrate one or more embodiments of the present invention and, together with the description, serve to explain the principles of the invention. The drawings are only for the purpose of illustrating one or more preferred embodiments of the invention and are not to be construed as limiting the invention. In the drawings:

FIG. 1 is an image of an agarose gel with the genotyping of codon 279 in the MMP-9 gene in cases and controls. The portion of the MMP-9 encompassing codon 279 A to G polymorphism was PCR amplified, digested with SmaI, and resolved by agarose gel electrophoresis. Subjects homozygous for 279Gln (194 bp product) (lanes 6 and 12); homozygous for 279Arg (172 bp product) (lanes 2, 5, 16, and 18); and heterozygous for 279 Gln/Arg (remaining lanes).

FIG. 2 is a plot of an example of the allelic discrimination results for the MMP-9 exon 6 polymorphism using the 7900HT system.

FIG. 3 is a table with the PCR primers and probes for detecting polymorphisms. (S)—sense primer; (AS)— antisense primer. The bolded and underlined letters are the SNPs designed in the probes for allelic discrimination.

FIG. 4 is an image of a gel of MMP-9 activity of media harvested from AALEB cells tested by zymography. Shown is media from AALEB cells transfected with the MMP-9 279Arg expression vector (lane 1), the MMP-9 279Gln expression plasmid (lane 2), nontransfected cells (lane 3), and from cells transfected only with the vector pCl-neo (lane 4).

FIG. 5 is an image of a gel of lysed bacterial cell extract, where cells were transformed with the MMP-9 279Gln (Q) and MMP-9 279Arg (R) expression vectors and induced to express the proteins by adding IPTG. At timepoints 1, 2, 4, and 8 hours following addition of IPTG, cells were harvested and lysed by ultra-sonication. Protein lysates were then analyzed by zymography for MMP-9 activity. Activity of MMP-9 279Gln (Q lanes) and MMP-9 279Arg (R lanes) at 1, 2, 4, and 8 hours are shown.

### DETAILED DESCRIPTION OF THE INVENTION

In one embodiment, the invention provides a method for determining the susceptibility of an individual, particularly a male, to COPD, by testing for the presence of an exon 6 codon 279 Gln/Arg SNP within the MMP-9 locus. The middle nucleotide in codon 279 may be adenine (“A”), such that the codon CAG codes for the amino acid glutamine (“Gln”), or may be the SNP wherein the middle nucleotide is guanine (“G”), such that the codon CGG codes for the amino acid arginine (“Arg”). The presence of the 279 arginine polymorphism indicates susceptibility to COPD, as hereafter described.

In the specification and claims, the following definitions are used:

An “oligonucleotide” can be DNA or RNA, and single-stranded or double-stranded. Oligonucleotides can be naturally occurring or synthetic, but are typically prepared by synthetic means.

The terms “nucleotide”, “base”, and “nucleic acid” can be used interchangeably. The terms “nucleotide sequence”, “nucleic acid sequence”, “nucleic acid molecule”, and “segment” are also interchangeable.

The term “primer” refers to a single-stranded oligonucleotide which acts as a point of initiation of template-directed DNA synthesis under appropriate conditions in an appropriate buffer and at a suitable temperature.

“Linkage” describes the tendency of genes, alleles, loci, or genetic markers to be inherited together as a result of their location on the same chromosome. It can be measured by percent recombination between two genes, alleles, loci, or genetic markers.

“Polymorphism” refers to the occurrence of two or more genetically determined alternative sequences or alleles in a population. A polymorphic marker or site is the locus at which divergence occurs. Preferred markers have at least two alleles, each occurring at a frequency of greater than 1%. A polymorphic locus may be as small as one base pair, such as a SNP. Polymorphic markers include restriction fragment length polymorphisms (RFLPs), variable number of tandem repeats (VNTRs), hypervariable regions, and microsatellites. The allelic form occurring most frequently in a selected population is sometimes referred to as the wild type or common form. Diploid organisms may be homozygous or heterozygous for allelic forms.

A single nucleotide polymorphism (SNP) occurs at a polymorphic site occupied by a single nucleotide, which is the site of variation between allelic sequences, and arises due to substitution of one nucleotide for another at the polymorphic site.

The terms “polymorphic position”, “polymorphic site”, “polymorphic locus”, and “polymorphic allele” are interchangeable and are defined as the location of a sequence identified as having more than one nucleotide represented at that location in a population comprising at least one or more individuals and/or chromosomes.

Nucleotides are sometimes referred to using standard single letter codes, and amino acid residues are sometimes referred to use standard three letter codes. Thus among DNA nucleotides “C” is cytosine, “T” is thymine, “A” is adenine, and “G” is guanine.

“COPD” is a general term that includes several overlapping lung conditions that share the common functional problem of airflow limitation, including but not limited to chronic bronchitis, emphysema, and asthma.

Matrix metalloproteinases (MMPs) comprise a family of at least 26 proteolytic, zinc-dependent endopeptidases that degrade extracellular matrix (ECM) in a substrate-specific manner and are classified according to their substrate specificity and structural similarities. An example is MMP-9.

MMPs are believed to be implicated in the pathogenesis of pulmonary emphysema through a proteinases-antiproteinase imbalance. The overall pattern of the ECM results from an intricate balance between the synthesis and degradation of its structural components. This continuous turnover and remodeling involves numerous degradative enzymes including a family of matrix MMPs that can degrade almost all of the ECM components. In addition, MMPs, in particular MMP-2 and MMP-9, have been implicated in the remodeling process of pulmonary airways (Swartz, M. A., et al. 2001. Mechanical stress is communicated between different cell types to elicit matrix remodeling. Proc Natl Acad Sci 98:6180-6185; Johnson, S., and A. Knox. 1999. Autocrine production of matrix metalloproteinase-2 is required for human airway smooth muscle proliferation. Am J Physiol 277:L1109-L 1117; Ye, S. 2000. Polymorphism in matrix metalloproteinase gene promoters: implication in regulation of the gene expression and susceptibility of various diseases. Matrix Biol 19:623-629). Although increased expression of MMPs has been observed in COPD, the possibility of polymorphisms in these genes being associated with this disease and thereby causing the dysregulated expression has not heretofore been investigated. These enzymes are classified according to their substrate specificity and structural similarities (Curran, S., and G. I. Murray. 1999. Matrix metalloproteinases in tumour invasion and metastasis. J Pathol 189(3):300-308; Nagase, H., and J. F. Woessner. 1999. Matrix metalloproteinases. J Biol Chem 274:21491-21494). Enhanced expression of MMPs can be detected in diseased tissues and plasma/sera from patients with various diseases such as cancer (Iizasa, T., et al. 1999. Elevated levels of circulating plasma matrix metalloproteinase 9 in non-small cell lung cancer patients. Clin Cancer Res 5(1):149-153; Lein, M., et al. 2000. Matrix-metalloproteinases and their inhibitors in plasma and tumor tissue of patients with renal cell carcinoma. Int J Cancer 85(6):801-804), osteoarthritis (Naito, K., et al. 1999. Measurement of matrix metalloproteinases (MMPs) and tissue inhibitor of metalloproteinases-1 (TIMP-1) in patients with knee osteoarthritis: comparison with generalized osteoarthritis. Rheumatology 38(6):510-515), multiple sclerosis (Lichtinghagen, R., et al. 1999. Expression of matrix metalloproteinase-9 and its inhibitors in mononuclear blood cells of patients with multiple sclerosis. J Neuroimmunol 99(1):19-26), and acute myocardial infarction (Kai, H., et al. 1998. Peripheral blood levels of matrix metalloproteases-2 and -9 are elevated in patients with acute coronary syndromes. J Am Coll Cardiol 32(2):368-372).

Regulation of transcription, activation of latent MMPs, and inhibition of MMP activity by TIMPs are the major factors that can influence the ultimate impact of MMPs on extracellular matrix degradation. It appears, however, that for most MMPs, the key step is transcriptional regulation, because most MMP genes are expressed only when active physiological or pathological tissue remodeling takes place (Matrisian, L. M. 1990. Metalloproteinases and their inhibitors in matrix remodeling. Trends Genet 6(4):121-125); Fini, M. E., et al. 1998. Proteolytic mechanisms in corneal ulceration and repair. Arch Dermatol Res 290(Suppl.):S12-S23).

MMP-9 can degrade native type IV and type V collagens, denatured collagens, entactin, proteoglycans, elastin and α₁-AT. MMP-9 plays a role in the migration of monocytes-macrophages and eosinophils to inflammatory foci by increasing the migration of these cells through the basement membrane, and in the disruption of the epithelial layer (Mautino, G., et al. 1999. Balance in asthma between matrix metalloproteinases and their inhibitors. J Allergy Clin Immunol 104(3 Pt 1):530-533). Sequence analysis of the MMP-9 gene has revealed a total of 10 variable sites, four in the promoter, five in the coding region (three of which alter the amino acid encoded), and one in the 3′ untranslated region (Zhang, B., et al. 1999. Genetic variation at the matrix metalloproteinase-9 locus on chromosome 20q12.2-13.1. Hum Genet 105(5):418-423). A variable length CA repeat located at −13 to −55 base pairs within the promoter region that affects promoter activity and creates a sequence-specific DNA-binding protein site has been described (Peters, D. G., et al. 1999. Functional polymorphism in the matrix metalloproteinase-9 promoter as a potential risk factor for intracranial aneurysm. Stroke 30(12):2612-2616). Promoter activity was reduced approximately by 50% when the lengths of the CA repeats were 14 to 21 compared to 22 or 23 length repeat. A SNP in the promoter region of MMP-9 also affects gene transcription (Zhang, B., et al. 1999. Functional polymorphism in the regulatory region of gelatinase B gene in relation to severity of coronary atherosclerosis. Circulation 99(14):1788-1794). A C→T polymorphism at −1562 results in higher transcription rates for the T allele than the C allele promoter. A recent study (Minematsu, N., et al. 2001. Genetic polymorphism in matrix metalloproteinase-9 and pulmonary emphysema. Biochem Biophys Res Commun 289:116-119) showed that the −1562 C/T transition of the MMP-9 gene is associated with the development of pulmonary emphysema induced by cigarette smoking. The size of the smoker population in this study was only 110 (45 with and 65 without emphysema). No difference in FEV₁ was found between the genotypes; however, emphysematous changes assessed by scoring the low attenuation area from chest CT-scans significantly differed between genotypes. These promoter polymorphisms may also be associated with COPD, because MMP-9 is expressed at higher levels in emphysematous compared to control lung tissue (Ohnishi, K., et al. 1998. Matrix metalloproteinase-mediated extracellular matrix protein degradation in human pulmonary emphysema. Lab Invest 78(9):1077-1087; Segura-Valdez, L., et al. 2000. Upregulation of gelatinases A and B, collagenases 1 and 2, and increased parenchymal cell death in COPD. Chest 117(3):684-694). The polymorphism in exon 6 is located in the catalytic domain of the MMP-9 enzyme and leads to a substitution of a positively charged amino acid (arginine) by an uncharged amino acid (glutamine), while the other alteration in amino acids is not likely to have such a profound change in enzyme activity.

COPD, which includes chronic bronchitis and emphysema, as well as asthma, is a debilitating disease characterized by airflow limitation that is not fully reversible and is usually progressive. Most COPD is attributable to cigarette smoking, although asthma and other causes of expiratory airflow obstruction can be contributing factors in some persons. The fact that only a minority of heavy smokers (20-35%) eventually develops symptomatic COPD suggests that the pathogenesis of this disease represents an interaction between genetic susceptibility and environmental exposures. It is not clear what causes a subgroup of smokers to have progressive disease, but the ultimate event is an irreversible loss of alveolar surface area and depletion of lung elastin. Many matrix metalloproteinases, including MMPs-2, -9, and -12, degrade elastin. The structure of the ECM is a result of a balance between the synthesis and degradation of its components, and the most widely accepted theory for the pathogenesis of pulmonary emphysema is a proteinase:antiproteinase imbalance. Studies of human samples with smoking-related emphysema show increases in many MMPs, including MMPs -1, -2, -8, -9, and -14. Immunoreactivity and mRNA levels for MMPs -2, -9 and -14 are found to be higher in emphysematous tissues compared to normal lung. Similarly, a deficiency in α₁-AT, which may result from degradation by MMP-9, increases susceptibility to developing COPD.

The inventors have found that there is a significantly increased risk for COPD in individuals, particularly men, homozygous for the SNP in exon 6 of the MMP-9 gene that changes glutamine to arginine in the active site of the enzyme. The invention thus relates to the discovery and utility of a portion of a gene, specifically the MMP-9 gene, having a nucleotide sequence and comprising a single nucleotide polymorphism at a specific position, in codon 279. The single nucleotide polymorphism results in a missense mutation.

SEQ ID NO:9 is the expressed MMP-9 protein, or matrix metalloproteinase-9, which constitutes an enzyme. The common variant has Gln at position 279, while the rare variant has Arg at position 279, resulting from a missense mutation, which alters the enzymatic activity of the MMP-9 protein, and increases the susceptibility to COPD. The MMP-9 protein is expressed by the MMP-9 gene, with the full-length gene shown at SEQ ID NO:10, wherein A at position 2665 is the common variant, resulting in expression of 279Gln, and G at the same position is the rare variant, resulting in expression of 279Arg. The cDNA that encodes for the protein is shown at SEQ ID NO:11, wherein A at position 841 is the common variant, resulting in expression of 279Gln, and G at the same position is the rare variant, resulting in expression of 279Arg.

The invention further provides allele-specific oligonucleotides that hybridize to a gene comprising the SNP in exon 6 of the MMP-9 gene or to the complement of the gene. Such oligonucleotides will hybridize to one polymorphic form of the nucleic acid molecule but not to other polymorphic forms of the sequence. Thus, such oligonucleotides can be used to determine the presence or absence of particular alleles of the polymorphic sequence described herein. These oligonucleotides can be probes or primers.

The invention further provides a method of analyzing a nucleic acid from an individual. The method allows the determination of whether the reference or the variant base is present at the polymorphic site. This type of analysis can be performed on a number of individuals, who are also tested (previously, concurrently or subsequently) for the presence of a disease phenotype. The presence or absence of a disease phenotype is then correlated with a base present at the polymorphic site in the individuals tested.

Thus the invention further relates to a method of predicting the presence, absence, or likelihood of the presence or absence of a particular phenotype or disorder associated with a particular genotype. The method comprises obtaining a nucleic acid sample from an individual and determining the identity of one or more bases (nucleotides) at specific (e.g., polymorphic) sites of nucleic acid molecules described herein, wherein the presence of a particular base at the site is correlated with a specified phenotype or disorder, thereby predicting the presence, absence, or likelihood of the presence or absence of the phenotype or disorder in the individual, wherein the phenotype or disorder is preferably COPD or a COPD-related disorder.

In one embodiment the present invention relates to isolated nucleic acid molecules comprising, or alternatively consisting of, a portion of the variant allele of the human MMP-9 gene. Preferably the invention employs an oligonucleotide of between about 10 and 50 base pairs, and more preferably between about 20 and 30 base pairs, which under suitably stringent conditions specifically hybridize to either the variant or common exon 6 codon 279 Gln/Arg SNP within the MMP-9 locus, or its reverse complement. Preferably the oligonucleotide specifically hybridizes to either the common sequence containing adenine at the exon 6 single nucleotide codon 279 Gln/Arg SNP, or alternatively to the variant sequence containing guanine at the exon 6 single nucleotide codon 279 Gln/Arg SNP. Particularly preferred are the following oligonucleotides, and oligonucleotides complementary thereto:

| | |
|---|---|
| CTCTACACCCGGGACGGCAATG; | (SEQ ID NO: 1) |
| and | |
| ACTCTACACCCAGGACGGCAATGC; | (SEQ ID NO: 2) |

wherein the bolded and underlined letters are the SNPs for allelic discrimination. In yet another embodiment, the oligonucleotide includes at least 10 base pairs comprising the SNPs for allelic discrimination, and oligonucleotides complementary thereto, such oligonucleotides including at least:

| | |
|---|---|
| TCTACACCCG; | (SEQ ID NO: 5) |
| GGGACGGCAA; | (SEQ ID NO: 6) |
| TCTACACCCA; | (SEQ ID NO: 7) |
| AGGACGGCAA; | (SEQ ID NO: 8) |

wherein the bolded and underlined letters are the SNPs for allelic discrimination.

Suitably stringent hybridization conditions must be employed such that an oligonucleotide specific for either the variant or common SNP hybridizes only thereto, and not to any other sequence or variant. Suitable hybridization conditions are well known in the art. Hybridization may be detected by means of a detectable label on the specific oligonucleotide, which label may be any known in the art, including without limitation enzymatic, radioactive, electroactive, fluorescent, chromagenic and other detectable labels.

In a preferred embodiment, the specific oligonucleotide is employed as a sequence specific probe. Such methods may also employ specific oligonucleotides as primers in an amplification reaction. For example, a portion of the exon 6 region of the MMP-9 gene containing the codon 279 Gln/Arg SNP may be subject to PCR using at least one primer that hybridizes with either the common or variant SNP, preferably at the 3′ terminal nucleotide of the primer, so that only fragments with the desired SNP will be amplified. Such techniques include amplification refractory mutation systems. Other methods may be employed, including various arrays systems, microplate array systems, and the like. In one embodiment, a MALDI-TOF based detection assay for SNPs may be used, such as that described by Blondal et al. (Blondal, T. et al. 2003. A novel MALDI-TOF based methodology for genotyping single nucleotide polymorphisms. Nucleic Acids Res 31 (24):e 155).

It may thus be seen that any method to determine the existence of the SNP may be employed, including methods that utilize gel electrophoresis as well as methods that do not. Similarly, while PCR or other amplification protocols are used in one embodiment, if assays with sufficient sensitivity are employed, it is also possible to detect the SNP without employing amplification protocols.

Where primers are employed for preferentially amplifying a sequence, the primer may be either a sense or antisense primer. Preferred nucleotide primers that may be so employed include either a 5′-TCTCCCCCTTTCCCACATC (SEQ ID NO:3) sense primer or a 5′-TGTGCTGTCTCCGCCTTCT (SEQ ID NO:4) antisense primer.

In yet another alternative embodiment of the invention, it is possible and contemplated to directly detect the MMP-9 polymorphism in protein; for example, the MMP-9 polymorphism can be isolated from blood leukocyte samples or other biological samples. Because the exon 6 codon 279 Gln/Arg SNP is part of a region that codes for expressed protein, the existence of the SNP may be determined by examination of the expressed protein.

The MMP-9 polymorphism, a change in a single amino acid, in one embodiment can be detected by purifying the MMP-9 protein with antibody affinity columns followed by mass spectrometric analysis of the purified protein. The mass spectrometry can utilize digestion with different proteases, such as trypsin or chymotrypsin, to generate small peptide fragments. The peptide containing the Gln amino acid can then be distinguished from the peptide containing Arg by the difference in mass.

In an alternative embodiment, one or more monoclonal antibodies may be produced by methods well known in the art which specifically and differentially recognize the antigenic peptide sequence with the 279Gln from that with the 279Arg. Antibody-based assays can utilize any immunoassay methodology, such as for example detection by western blot analysis or detection by ELISA.

In one embodiment, the invention relates to a method for predicting the likelihood that an individual will have a disorder associated with a G at the nucleotide position corresponding to the second position in codon 279 of exon 6 of the gene encoding MMP-9 (or diagnosing or aiding in the diagnosis of such a disorder) comprising the steps of obtaining a DNA sample from an individual to be assessed and determining the nucleotide present at the second position of codon 279 of exon 6 of the gene encoding MMP-9. The presence of a G at this position, particularly if the individual is homozygous for G, indicates that the individual has a greater likelihood of having a disorder associated therewith than an individual having an A at the position.

Conversely, the invention relates to a method for predicting the likelihood that an individual will have a disorder associated with an A at the nucleotide position corresponding to the second position in codon 279 of exon 6 of the gene encoding MMP-9 (or diagnosing or aiding in the diagnosis of such a disorder) comprising the steps of obtaining a DNA sample from an individual to be assessed and determining the nucleotide present at the second position of codon 279 of exon 6 of the gene encoding MMP-9. The presence of an A at this position, whether homozygous for A or heterozygous, indicates that the individual has a lesser likelihood of having a disorder associated therewith than an individual having a homozygous G at the position.

Representative disorders that may be detected, diagnosed, identified, treated, prevented, and/or ameliorated by the present invention include COPD, including chronic bronchitis, emphysema, and asthma.

It may thus be seen that detection of the MMP-9 form in individuals can be employed to develop simple diagnostic tests to determine individuals who are at higher risk for developing COPD. This detection may be employed for several purposes. First, individuals with the MMP-9 polymorphism can be warned of their increased susceptibility and may be encouraged to stop smoking. Second, individuals with higher risk can be advised to develop a habit of diet (increased intake of foods with high contents in anti-oxidants) that could reduce the development of emphysema. Third, individuals may be advised to more often have a physician test their pulmonary function, such as for example twice yearly, and receive appropriate medical advice on how to avoid a steep decline in pulmonary function. It may be seen that these measures may lead to decreased public health burden. In general, information obtained by the methods of this invention, when used alone or in combination with other genetic or clinical data, allow an accurate assessment of prognosis and treatment options for COPD.

In addition to providing for a variety of diagnostic measures, as set forth above, knowledge of the MMP-9 polymorphism can also be used to directly test the role of this enzyme in the pathogenesis of emphysema. Data indicate that MMP-9 may be the major enzyme that sets the degradation of elastin in motion. Therefore, inhibition of this MMP-9 polymorphism provides a very useful therapeutic avenue for reducing progression of COPD. Peptidomimetic compounds, many of which consist of modified tripeptides, inhibit MMP-9 activity. Such inhibitors, or other inhibitors that are specific to the MMP-9 polymorphism, would decrease its activity and thereby reduce the formation of emphysematous lung.

Thus the MMP-9 SNP described herein may be used to screen or select patients for particularly therapeutic regimens, particularly therapeutic regimens wherein the MMP-9 SNP is relevant to the treatment protocol. Thus patients for treatment with high doses of anti-oxidants may be selected for treatment based on testing for the MMP-9 SNP. Similarly, patients for treatment with MMP-9 inhibitors may be selected based testing for the MMP-9 SNP.

It is also possible and contemplated that testing for the MMP-9 SNP may be employed in drug discovery and development. Thus the exon 6 codon 279 Gln/Arg SNP within the MMP-9 locus may itself serve as a drug target. Alternatively, a specific inhibitor of the exon 6 codon 279 Gln/Arg SNP within the MMP-9 locus may be developed based on knowledge of such SNP, and by means of assays and tests incorporating the sequences, primers and probes disclosed herein.

The discovery that MMP-9 Gln/Arg polymorphism is associated with COPD further enables research on understanding the functional role of the polymorphism in this disease. The two variants have been successfully expressed in both mammalian and bacterial expression systems. The mammalian expression system has been used to express the two variants in a cell line that normally does not produce MMP-9. The expressed protein is secreted from this cell line and the media that contains the expressed variants can be tested for differences in enzyme activities toward various substrates. Purified forms of enzyme variants are similarly produced by recombinant means, including use of a bacterial expression plasmid containing a Thioredoxin-Histidine tag. This tag forms a metal-binding domain, which allows the purification of the expressed protein from bacterial Esherishia coli extracts. The purified enzyme variants are then tested for differences in their activity toward MMP-9 substrates, including gelatin, elastin, and α₁-AT (alpha-1-antitrypsin).

It is thus also possible and contemplated that the two variant forms of the expressed protein may be made, such as by recombinant means, and independently tested for substrate activity. Thus compounds may be screen against both the 279 Gln MMP-9 protein and the 279 Arg MMP-9 protein, and differences in activity with respect to compounds accordingly determined.

It is further possible and contemplated that expression systems can be used to screen for inhibitors of the rare variant which do not affecting the common variant of the enzyme. Specific inhibitors for the rare MMP-9Arg variant are potential drugs to decrease the risk of developing COPD or reduce the progression of this disease in the high-risk population.

### INDUSTRIAL APPLICABILITY

The invention is further illustrated by the following non-limiting examples.

### EXAMPLE 1

### COPD Definition

COPD was defined by a FEV₁/FVC<70% as measured by spirometry. These criteria are similar to those suggested as part of the GOLD criteria (Pauwels, R. A., et al. 2001. Global strategy for the diagnosis, management, and prevention of chronic obstructive pulmonary disease. NHLBI/WHO Global Initiative for Chronic Obstructive Lung Disease (GOLD) Workshop summary. Am J Respir Crit Care Med 163(5):1256-76) and were used in the Lung Health Study (Anthonisen, N. R., et al. 1994. Effects of smoking on intervention and use of an inhaled anticholinergic bronchodilator on the rate of decline of FEV₁. The Lung Health Study. JAMA 272:1497-1505). Participants who met the criteria for diagnosis of COPD at cohort entry were classified as prevalent cases. Repeat testing within a 1-year period has been performed on 189 participants from the Veterans'Cohort and 171 participants from the Lovelace Women's Cohort with 90 and 95% concordance of those initially classified with chronic airflow obstruction, respectively. These tests established chronicity of airflow limitation through demonstration of FEV₁/FVC<70% by spirometry that was not reversed into the normal range after a bronchodilation (Anthonisen, N. R., et al., supra). Patient-reported doctor-diagnosed asthma was used to identify asthma patients (Samet, J. M. 1987. Epidemiologic approaches for the identification of asthma. Chest 91(6 Suppl):74S-78S). Participants (17 from the Veterans' and 11 from the Women's cohorts) who had airway obstruction with a history of doctor's diagnosed asthma and an FEV₁/FVC 65-70% could have a reversible airway obstruction due to asthma and, therefore, were excluded from initial analysis of associations between allelotype and COPD.

### EXAMPLE 2

### Genotyping of Polymorphisms in MMP-9

Genomic DNA was isolated from peripheral blood lymphocytes by digestion with proteinase K in sodium dodecyl sulfate (1%), followed by standard phenol/chloroform extraction and ethanol precipitation as described (Sambrook, J., et al. 1989. Molecular Cloning. A Laboratory Manual. 2nd ed.). The sequence flanking the polymorphic region of each SNP was amplified by PCR using 10 ng of DNA. Negative controls without DNA template were included with each set of reactions. Several methods of genotyping were used to verify the obtained results. Restriction fragment length polymorphism was performed on 50-100 samples to verify the results obtained by the allelic discrimination assay. This assay integrates PCR and detection using allelic discrimination, which applies allele-specific TaqMan probes, a two-dye reporter system, and a reference dye. Wild-type and variant reporter sequences were discriminated using dual labeled reporters and by using mismatched sequences to validate the SNP-genotype. To further ensure quality control, DNA samples from 20 participants whose allelotypes were known were included at random within each of the 96-well plates of the allelic discrimination assay. In addition, approximately 10% of the samples were reexamined by a different person. All analyses were performed blinded with respect to participants' characteristics. These controls showed 100% concordance with expected results.

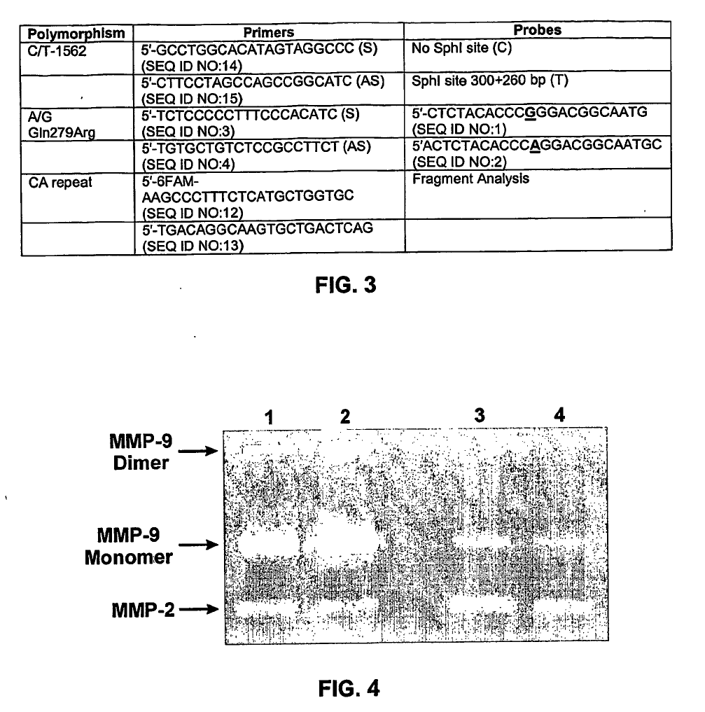

The primers (SEQ ID NO:3, SEQ ID NO:4) and probes (SEQ ID NO:1, SEQ ID NO:2) employed for allelic discrimination are shown in FIG. 3. The C→T polymorphism was analyzed using a restriction length polymorphism (RFLP) assay, because several attempts to design primers and probes for this polymorphism failed. The presence of the rare T allele introduces a restriction site for SphI, which is absent when the common C allele is present.

The number of CA repeats in the promoter region of MMP-9 was determined by PCR using a fluorescently labeled 6-FAM forward primer (SEQ ID NO:12) and an unlabeled reverse primer (SEQ ID NO:13). The alleles were separated by a laser-based automated DNA sequencer, ABI PRISM 3100. The product peaks were visualized using GenescanView software (Bio Molecular Research, Padova, Italy). The size of the PCR products was calculated from a standard curve made from internal standards. DNA standards containing 14, 21, 22, and 23 CA repeats were identified by direct sequencing and were included in each experiment as positive controls.

### EXAMPLE 3

### Identification and Detection of Nucleotide Polymorphism in MMP-9 Gln279Arg

The following illustrates two methods that were used to identifying nucleotide polymorphisms and for genotyping individuals.

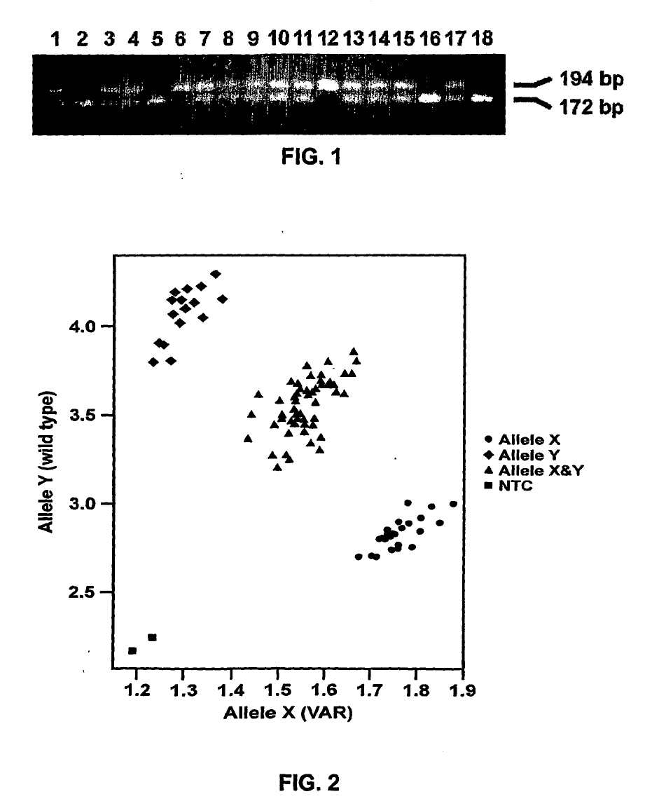

One method of allelotyping for the codon 279 polymorphism relied on the loss of a SmaI restriction site due to the polymorphism. Thus, the frequency for the codon 279 A→G polymorphism was determined by polymerase chain reaction (PCR) and restriction fragment length polymorphism (RFLP), a common approach for genotyping. PCR primer sequences have been described (Zhang, B., et al. 1999. Genetic variation at the matrix metalloproteinase-9 locus on chromosome 20q12.2-13.1. Hum Genet 105(5):418-423). Amplification product of 194 base pairs was generated. The PCR product was digested overnight with SmaI, and the DNA fragments were resolved on an agarose gel. As shown in FIG. 1, three situations can be seen: (1) a single band of 194 base pairs indicating homozygosity for the rare allele; (2) two different bands of 194 and 172 base pairs indicating heterozygosity for the rare and common alleles; and (3) a single band of 172 base pairs indicating homozygosity for the common allele.

Another approach used the ABI PRISM 7900 HT Sequence Detection System (Applied Biosystems, Foster City, Calif.) to detect the G and A polymorphisms using TaqMan probes. In a two-allele system, probes for each allele are multiplexed in a single tube with the DNA samples. This method allows a rapid allelotyping, because of the specificity of the probes during the amplification procedure. Therefore, no further handling of the samples is necessary. Two different reporter dyes were used to label the probes, so that the one dye (VIC) gives maximum signal for the A allele and the other dye (FAM) gives maximum signal for the G allele, as shown in FIG. 2. The heterozygote samples give an intermediate signal for both, also as shown in FIG. 2.

A total of 1067 DNA samples were analyzed using the allelic discrimination assay. To further ensure quality control, DNA samples from 20 subjects whose allelotypes were known were included at random within each of the 96-well plate of the allelic discrimination assay. In addition, approximately 10% of the samples were reexamined by a different person. All analyses were performed blinded with respect to subject characteristics. The A and the G alleles were represented in 65% and 35% of the smoker population analyzed.

### EXAMPLE 4

### Statistical Analysis

All analyses were conducted separately by cohort. Summary information by COPD status within each cohort was examined for demographic variables (age, gender, ethnicity); clinical risk factors (current smoking status, duration and pack years of cigarette smoking); and polymorphism status (homozygous-rare, heterozygous, homozygous-common). Because of the small number of women in the Veterans'Cohort (1 COPD and 29 non-COPD), women in this cohort were excluded from all analyses. Fisher's exact test was used to compare gene polymorphism frequencies between participants with and without COPD.

Distributions of genotypes were assessed for Hardy-Weinberg equilibrium using a generalization of Fisher's exact test (Guo, S., and E. Thompson. 1992. Performing exact tests of Hardy-Weinberg proportion for multiple alleles. Biometrics 48:361-372). All pairs of loci (T-1562, 279Arg, and CA repeats) were tested for linkage disequilibrium using likelihood tests and permutation procedures to estimate significance levels (Goudet, J., et al. 1996. Testing differentiation in diploid populations. Genetics 144:1933-1940; Slatkin, M., and L. Excoffier. 1996. Testing for linkage disequilibrium using the EM algorithm. Heredity 76:377-383) as implemented in Arlequin v2.0 (Schneider, S., et al. 2000. A software for population genetics data analysis: Genetics and Biometry Laboratory, University of Geneva, Switzerland). The degree of disequilibrium were described using D′ (Lewontin, R. C. 1964. The interaction of selection and linkage. I. General considerations; heterotic models. Genetics 49:49-67; Zapata, C., C. Carollo, and S. Rodriguez. 2001. Sampling variance and distribution of the D′ measure of overall gametic disequilibrium between multiallelic loci. Ann Human Gen 65:395-406).

Logistic regression models were fit to estimate odds ratios (OR) for COPD and to allow adjustment for multiple covariates. Effects for a range of genetic models including dominant, codominant, and recessive models for the variant alleles were estimated. Covariates in all models included age, ethnicity, sex (Veterans'Cohort only), and smoking information (former or current, or the continuous forms of smoking duration and pack-years). Logistic regression models for each polymorphism were fit and the indicators were included as predictors for only those polymorphisms at one region plus the covariates. Since there was linkage disequilibrium between some of the polymorphisms, the polymorphisms were examined separately and models did not include more than one polymorphism. Interactions between the genotypes and each covariate, especially cigarette smoking (duration and pack years), and interactions between the covariates also were investigated.

Linear regression models were used to assess the association between the genetic predictor variables and the FEV₁/FVC ratio. Covariates and interactions were included in the modeling as done for the logistic regression models above. Effects were summarized using least squares means and 95% confidence intervals. Fisher's exact tests and the linear and logistic regression modeling were conducted using Statistical Analysis System SAS v8.02. (SAS Institute, Inc., Cary, N.C.).

The association between estimated haplotype frequencies and COPD was evaluated with a two-stage process. First, haplotype frequencies based on T-1562, 279Arg, and CA repeats were estimated using Stram's implementation (Stram, D. O., et al. 2003. Choosing haplotype-tagging SNPS based on unphased genotype data using a preliminary sample of unrelated subjects with an example from the Multiethnic Cohort Study. Hum Hered 55(1):27-36) of a partition-ligation-expectation-maximization algorithm (Qin, Z. S., T. Niu, and J. S. Liu. 2002. Partition-ligation-expectation-maximization algorithm for haplotype inference with single-nucleotide polymorphisms. Am J Hum Genet 71(5):1242-1247). For each possible haplotype, h, an estimate was made of the number of copies of h contained in the true pair of haplotypes carried by that individual conditional on the observed genotype and assuming Hardy-Weinberg equilibrium. The expected haplotype frequencies are analogous to haplotype dosages and were used in the second step as independent variables in logistic regression models that also included adjustment for age, smoking duration, smoking status, ethnicity, and gender when appropriate. Haplotype dosages for each participant were calculated separately for Non-Hispanic Whites and Hispanics, and then separate logistic regression models were fit for each cohort and for each cohort and ethnicity. Furthermore, within each cohort separate logistic regression models were fit by ethnic group. Likelihood ratio tests were used to test whether haplotype dosages were associated with risk of COPD and Wald's Chi-square tests were used to evaluate whether individual haplotypes were different from the reference haplotype.

### EXAMPLE 5

### Study Population

Study participants were drawn from two ongoing cohort studies: the Veterans'Cohort (N=597) and the Lovelace Women's Cohort (N=411). Participants of the Veterans'Cohort were recruited from the New Mexico Veterans' Health Care System, which provides medical care for approximately 35,000 veterans in New Mexico, west Texas, and southern Colorado. Because women are underrepresented in most COPD cohorts, including the Veterans'Cohort, 411 current and former women smokers were recruited from the metropolitan area of Albuquerque. Inclusion criteria for both cohorts included age 40 to 75 years and a history of cigarette smoking (former and current smokers were included). Inclusion criteria for minimum smoking history differed by cohort (≧100 cigarettes and ≧20 pack years for the Veterans' and Women Cohorts, respectively). Informed consent was obtained from each participant prior to completing a standardized clinical examination including spirometry and a standardized questionnaire that provided detailed information on demographics, smoking history, and respiratory health. This study was approved by the Institutional Review Boards of the Lovelace Respiratory Research Institute, the New Mexico Veterans Health Care System, and the University of New Mexico Health Sciences Center.

The two cohorts are summarized by COPD status as shown on Table 1:

TABLE 1

| | VA (n = 597) | | Lovelace (n = 411) | |
|---|---|---|---|---|
| Cohort | COPD | Non-COPD | COPD | Non-COPD |
| N (%) | 163 (27) | 434 (73) | 128 (31) | 283 (69) |
| Age Class (years) N (%) | | | | |
| 40-49 | 8 (4.9) | 75 (17.2) | 15 (11.7) | 95 (33.6) |
| 50-59 | 44 (27.0) | 186 (42.9) | 28 (21.9) | 99 (35.0) |
| 60-69 | 69 (42.3) | 118 (27.2) | 50 (39.1) | 58 (20.5) |
| ≧70 | 42 (25.8) | 55 (12.7) | 35 (27.3) | 31 (11.0) |
| Gender N (%) | | | | |
| Male | 162 (99) | 405 (93) | 0 | 0 |
| Ethnicity N (%) | | | | |
| Non-Hispanic White | 110 (67.5) | 243 (56.0) | 116 (90.6) | 225 (79.5) |
| Hispanic | 31 (19.0) | 135 (31.1) | 10 (7.8) | 45 (15.9) |
| Other | 22 (13.5) | 56 (12.9) | 2 (1.6) | 13 (4.6) |
| Current Smoker Yes/No (%) | 60/100 (37.5) | 140/284 (33.0) | 57/71 (44.5) | 168/115 (59.4) |
| Smoking, Duration (years) | 36.3 | 27.0 | 40.5 | 33.4 |
| average (min, max) | (1.0-60.0) | (0.5-60.0) | (15.0-59.0) | (10.0-61.0) |
| Smoking, pack years | 52.6 | 32.5 | 56.2 | 42.9 |
| average (min, max) | (0.10-220.0) | (0.2-140.0) | (20.0-184.8) | (20.0-167.8) |

The prevalence of COPD was 27% and 31% within the Veteran and the Women's Cohorts, respectively. Overall the age of participants with COPD were older than non-COPD participants in both cohorts, as expected for a disease that increases in frequency with age. The median age for subjects with and without COPD was 65 and 57 years for the Veterans'Cohort (p<0.001), respectively, and 63 and 55 years for the Women's Cohort. The Veterans'Cohort had fewer participants in the youngest age group than the Women's Cohort (p<0.001). The Veterans'Cohort was 95% male. The ethnic distribution differed between the Veterans'Cohort and the Women's Cohort (p<0.001); the percentage of non-Hispanic White participants was higher in the Women's Cohort, while the Veterans'Cohort had a higher percentage of Hispanics and other ethnic groups.

In the Women's Cohort a higher percentage of non-COPD subjects were current smokers compared to those who had COPD (p=0.01), while in the Veterans'Cohort, the percentages of current smokers was similar for subjects with and without COPD. In general, the Women's Cohort had a higher rate of current smokers (54%) than the Veterans'Cohort (35%) (p<0.001). In both cohorts, a higher proportion of non-Hispanic white participants had COPD than Hispanic participants (p<0.001).

Average smoking duration and pack years were greater for participants with COPD than for participants without COPD for both cohorts (p<0.001). Furthermore, in both cohorts, the OR for COPD increased by factors of 1.7 and 2.0 for every 10 additional years of smoking duration and age, respectively (data not shown). Finally, COPD was significantly associated with phlegm production when adjusted for age and smoking duration in the Veterans'Cohort (OR=2.0, 95% Cl=1.4-3.0; p<0.001) but not in the Women's Cohort. These basic demographic and clinical risk factors are similar to those reported in other studies of COPD.

### EXAMPLE 6

### Distribution of Genotype by Cohort

The SNPs were determined in DNA samples from 597 study participants from the Veterans' Cohort and 411 study subjects from the Women's Cohort as shown in Table 3. Because the frequency distribution of the CA repeat polymorphism showed a bimodal distribution of alleles (data not shown), the alleles were divided into two subclasses: CA repeats ≦16 and ≧17 were designated as Short and Long, respectively, and treated similarly to genotype data. The distributions of homozygotes and heterozygotes for each polymorphism in each ethnic group and cohort and each COPD/non-COPD group within cohorts conformed to expectations under the assumption of Hardy-Weinberg equilibrium. The 279Arg was in linkage disequilibrium with both T-1562 (p<0.001) and CA repeat (p<0.001). As a measure for linkage disequilibrium, D′ was determined, as shown in Table 2. The confidence intervals for D′ are wide because the variant combinations are rare; however, based on the Gabriel criteria, the C-1562T/Gln279Arg appear to be in the same haplotype block and although there was considerable linkage, the CA repeat does not appear to be in the same haplotype block with Gln279Arg. Of particular note, all participants with the homozygote rare T-1562 (N=23) had rare 279Arg. However, many subjects that were homozygote for 279Arg had the C-1562.

TABLE 2

| Linkage comparison | D prime | 95% Cl* |
|---|---|---|
| C-1562T - Gln279Arg | 0.99 | 0.96-1.00 |
| Gln279Arg - CA repeat | 0.90 | 0.86-0.95 |
| C-1562T - CA repeat | 0.97 | 0.91-1.00 |

TABLE 3

| Veterans' Cohort | | | | | | | | Lovelace Cohort | | | | | |
|---|---|---|---|---|---|---|---|---|---|---|---|---|---|
| | | | | Non-Hispanic | | | | | | Non-Hispanic | | | |
| | | | | White | | Hispanic | | ALL | | White | | Hispanic | |
| | | ALL | | | Non- | | Non- | | Non- | | Non- | | Non- |
| | | COPD | Non-COPD | COPD | COPD | COPD | COPD | COPD | COPD | COPD | COPD | COPD | COPD |
| G1n279Arg | Common | 36.7 | 51.6 | 35.2 | 45.3 | 48.4 | 60.9 | 39.8 | 38.2 | 37.1 | 36.3 | 70.0 | 50.0 |
| | Heterozyg. | 45.3 | 40.7 | 45.4 | 44.4 | 41.9 | 35.2 | 46.9 | 51.1 | 49.1 | 51.6 | 30.0 | 45.5 |
| | Rare | 18.0*** | 7.7 | 19.4* | 10.2 | 9.7 | 3.9 | 13.3 | 10.7 | 13.8 | 12.1 | 0.0 | 4.5 |
| | n | 161 | 405 | 108 | 225 | 31 | 128 | 128 | 280 | 116 | 223 | 10 | 44 |
| C/T-1562 | Common | 70.1 | 76.7 | 67.3 | 72.3 | 85.7 | 86.1 | 76.6 | 72.0 | 76.7 | 68.6 | 70.0 | 90.9 |
| | Heterozyg. | 25.3 | 21.2 | 26.9 | 24.9 | 14.3 | 13.9 | 20.3 | 26.5 | 19.8 | 29.6 | 30.0 | 9.1 |
| | Rare | 4.6 | 2.1 | 5.8 | 2.8 | 0.0 | 0.0 | 3.1 | 1.4 | 3.4 | 1.8 | 0.0 | 0.0 |
| | n | 154 | 390 | 104 | 217 | 28 | 122 | 128 | 279 | 116 | 223 | 10 | 44 |
| CA repeat | SS | 24.0 | 25.4 | 30.5 | 34.0 | 12.9 | 14.1 | 29.7 | 24.8 | 30.2 | 28.1 | 20.0 | 11.1 |
| | LS | 47.5 | 48.2 | 45.7 | 47.6 | 54.8 | 51.2 | 48.4 | 52.8 | 49.1 | 52.2 | 50.0 | 62.2 |
| | LL | 28.5 | 26.5 | 23.8 | 18.5 | 32.3 | 34.7 | 21.9 | 22.3 | 20.7 | 19.6 | 30.0 | 26.7 |
| | n | 158 | 378 | 105 | 206 | 31 | 121 | 128 | 282 | 116 | 224 | 10 | 45 |

P-value is for Fisher's Exact Test that genotype distributions are the same for cases and controls. The difference in COPD and Non-COPD subjects (***p < 0.001, *p < 0.05) is also indicated. Distribution of genotypes in COPD and non-COPD participants by cohort (%).

The distribution of Gln279Arg genotypes in the Veterans'Cohort was significantly different between COPD and non-COPD participants. A higher prevalence of the 279Arg homozygote genotype (18.0% vs. 7.7%, p<0.001) and the heterozygote genotype (45.3% vs. 40.7%, p<0.05) was seen in participants with COPD compared to non-COPD participants. The distribution of the homozygote rare 279Arg was significantly different within the Non-Hispanic White participants. This trend in difference was recapitulated within the Hispanic participants, but was not significant due to the lower number of Hispanic participants with COPD. The rare T-1562 allele and the CA repeat lengths were distributed similarly among the participants with and without COPD in the Veterans'Cohort. No statistical differences in distribution for any of the genotypes were observed between COPD and non-COPD participants in the Women's Cohort. However, among the non-COPD participants there was a significant difference in the distributions between the Women's Cohort and the Veterans'Cohort, with the prevalence of the homozygote common allele being higher in the Veterans'Cohort (Table 3).

Analysis of the Veterans'Cohort after excluding all participants with less than 20 pack years did not alter the distribution of 279Arg or the significance. Specifically, the percentage of participants with 279Arg was increased from 18% to 18.2% in the Veterans'Cohort and within the Non-Hispanic White participants from 19.4 to 20.6%.

### EXAMPLE 7

### Association of Allelotype with COPD

The rare 279Arg variant was significantly associated with COPD within the Veterans'Cohort, as shown in Table 4. After adjustment for age, smoking duration, current smoking status, and gender (for Veterans'Cohort only) the OR for participants with COPD who were homozygous for 279Arg variant was 3.5 relative to those who were homozygous for the common allele (p<0.001). In addition, participants who were heterozygous also showed a significant increased risk for the disease (OR=1.6; p<0.05), indicating a codominant association between 279Arg and COPD. This association was significant within the Non-Hispanic White but not within the Hispanic participants. A marginal significant association between COPD and the homozygous rare T-1562 within the Non-Hispanic White participants in the Women's Cohort was observed after adjustment for age, smoking duration, and current smoking status (OR=1.4, p=0.06).

TABLE 4

| Veterans' Cohort | | | | | Women's Cohort | | |
|---|---|---|---|---|---|---|---|
| | | | Non-Hispanic | | | Non-Hispanic | |
| | | All | White | Hispanic | All | White | Hispanic |
| G1n279Arg | n | 553 | 327 | 152 | 408 | 339 | 54 |
| | Heterozyg. | 1.6 (1.0-2.5) | 1.6 (0.9-2.7) | 1.3 (0.5-3.2) | 0.9 (0.5-1.5) | 0.9 (0.6-1.6) | 0.6 (0.1-3.1) |
| | Homozyg. | 3.5 (1.8-6.5)*** | 2.5 (1.2-5.5)** | 2.9 (0.5-15.2) | 1.1 (0.5-2.3) | 1.1 (0.5-2.3) | NA^{a} |
| | Rare | | | | | | |
| C/T-1562 | n | 532 | 315 | 144 | 407 | 339 | 54 |
| | Heterozyg. | 1.2 (0.7-1.9) | 1.2 (0.7-2.2) | 0.7 (0.2-2.4) | 0.6 (0.3-1.1) | 0.5 (0.3-0.9) | 1.3 (0.2-8.2) |
| | Homozyg. | 2.9 (0.9-9.2) | 2.6 (0.7-9.5) | NA^{b} | 1.7 (0.4-7.6) | 1.4 (0.3-6.4)* | 5.4 (0.8-36.0) |
| | Rare | | | | | | |
| CA repeat | n | 525 | 307 | 145 | 410 | 340 | 55 |
| | Long-Short^{c} | 1.0 (0.6-1.7) | 1.1 (0.6-2.1) | 1.2 (0.3-4.9) | 0.8 (0.5-1.4) | 1.0 (0.5-1.7) | 0.4 (0.1-3.2) |
| | Long-Long^{c} | 1.2 (0.7-2.0) | 1.4 (0.7-2.9) | 0.9 (0.2-4.3) | 0.9 (0.5-1.7) | 1.0 (0.5-2.1) | 0.8 (0.1-7.6) |

Provides association of genotypes within the cohorts to COPD.
NA^{a} means not applicable due to small sample size (maximum likelihood is not achieved);
NA^{b} means not applicable due to zero participants with homozygous rare. Adjusted ratios are for genotypes for the co-dominant genetic model. Models include an adjustment for current smoking status and linear adjustments for age and smoking duration.
^{c}OR (95% Cl) relative to homozygous common and Short—Short CA repeats.
*p < 0.06
**p < 0.05
***p < 0.001.

All models showed that smoking variables, including duration, pack years, and current status were significantly associated with COPD, but none of the variables was a cofounder with the genetic variables. Interactions with 279Arg and the smoking variables that differed between the two cohorts (e.g., pack years <20 and current status) were examined but no significant interactions or confounding was found. There were no genotype interactions with any of the smoking variables. Analysis of the Veterans'Cohort after excluding all participants with less than 20 pack years did not affect the significant association to increased risk of COPD in participants with 279Arg (OR was increased to 3.8 for all participants and to 2.9 for the Non-Hispanic White participants). Inclusion of the participants who had doctor-diagnosed asthma with an FEV₁/FVC 65-75% (17 from the Veterans' and 11 from the Women's cohorts) did not change these results.

The rare 279 Arg variant was also significantly associated with FEV₁/FVC within the Veterans' Cohort. Within the Veterans'Cohort, participants with the two 279Arg variants showed a decrease of 3.4% (95% Cl=0.6%-6.3%) in FEV₁/FVC ratio after adjusting for age, smoking duration, current smoking status, and gender (p=0.02). Participants with one 279Arg variant showed a decrease of 2.2% (95% Cl=0.4%-4.0%, p=0.02). No association was found for 279Arg with FEV₁/FVC within the Women's Cohort. In addition, participants within the Women's Cohort with one or two 279Arg variants showed no significant differences relative to participants with no 279Arg variant. Similar results were obtained when the participants with a history of doctor's diagnosed asthma and an FEV₁/FVC 65-70% were included.

### EXAMPLE 8

### Haplotype Analyses

Haplotype (common [C], rare [R], long [L], short [S]) frequencies formed from MMP-9 C/T-1562, MMP-9 Gln279Arg, and CA repeats were estimated for each cohort and evaluated for differences with respect to COPD status, as shown in Table 5. Distributions of haplotypes were different between COPD and non-COPD participants in the Veterans'Cohort (p=<0.001) but not in the Women's Cohort (p=0.17). ORs adjusted for age, smoking duration, current smoking status, and gender (for Veterans'Cohort only) were calculated relative to the common C-1562, Gln279, and short CA repeats (C-C-S) for haplotypes with frequencies >0.005 (Table 5). The haplotypes with significant association with COPD were C-R-L (1.6, 95% Cl, 1.1-2.5) and R-R-L (1.6, 95% Cl, 1.0-2.6) compared to the C-C-S haplotype. The fact that participants with a T-1562 with 279Arg haplotypes showed the same OR as participants with the C-1562 with 279Arg haplotype indicates that the 279Arg polymorphism is the main determinant for the increased association by MMP-9 and risk for COPD. Analyses were performed separately on the four cohort×ethnicity combinations (data not shown). Models with and without covariates were compared using likelihood ratio tests. Models with haplotype dosages were no better than covariate only-models for predicting COPD status of Hispanic Veterans and of women. Haplotype information was useful for predicting COPD in non-Hispanic White Veterans, however the only adjusted OR different from the reference haplotype, C-C-S, was C-C-L (p=0.05, OR=0.4, 95% Cl=0.2-1.0).

TABLE 5

| | Veterans' Cohort | | | | | Women's Cohort | | | | |
|---|---|---|---|---|---|---|---|---|---|---|
| | Relative | | | Adjusted | | Relative | | | Adjusted | |
| | Frequency | | | OR^{a} | | Frequency | | | OR^{b} | |
| | | Non- | COPD | Estimate | P- | | Non- | COPD | Estimate | P- |
| Haplotype | COPD | COPD | (%) | 95% Cl | value | COPD | COPD | (%) | 95% Cl | value |
| C-C-S | 0.464 | 0.491 | 26.1 | 1.0 | | 0.535 | 0.500 | 32.6 | 1.0 | |
| C-C-L | 0.125 | 0.221 | 17.5 | 0.7 | 0.15 | 0.091 | 0.132 | 23.8 | 0.6 | 0.11 |
| | | | | (0.5-1.1) | | | | | (0.3-1.1) | |
| C-R-S | 0.010 | 0.000 | 100.0 | | | 0.004 | 0.006 | 24.0 | | |
| C-R-L | 0.225 | 0.161 | 34.4 | 1.6 | 0.02 | 0.237 | 0.213 | 33.4 | 1.0 | 0.94 |
| | | | | (1.1-2.5) | | | | | (0.6-1.5) | |
| R-C-S | 0.000 | 0.000 | | | | 0.000 | 0.005 | 0.0 | | |
| R-C-L | 0.000 | 0.003 | 0.0 | | | 0.007 | 0.000 | 100.0 | | |
| R-R-S | 0.005 | 0.003 | 34.9 | | | 0.000 | 0.000 | | | |
| R-R-L | 0.171 | 0.121 | 34.6 | 1.6 | 0.05 | 0.126 | 0.144 | 28.4 | 1.1 | 0.78 |
| | | | | (1.0-2.6) | | | | | (0.6-2.0) | |

In Table 5, which provides MMP-9 C/T-1562, Gln279Arg, and CA repeat haplotype frequencies within the Veteran's and Women's Cohort, the following notes apply:
^{a}Logistic model adjustments included age, smoking duration, current smoking status, and gender.
^{b}Logistic model adjustments included age, smoking duration, and current smoking status.
^{a}For C-1562T and Exon 6, C = common and R = rare; for CA repeat length, S = Short and L = Long.

### EXAMPLE 9

### Expression of MMP-9 Gln279Arg Polymorphism in Mammalian Cells

The 279Gln common variant of MMP-9 and the 279Arg rare variant were expressed in a mammalian expression system. cDNA encoding MMP-9 cloned in BlueScript plasmid was obtained from the University of Washington, School of Medicine. This cDNA (corresponding to SEQ ID NO:11) was cloned into the pCl-Neo mammalian expression vector. Analysis of the sequence by restriction using SmaI showed that the cDNA sequence contained the rare G allele encoding for 279Arg. Two primers, GAGAGACTCT ACACCC A GGACGGCAATGCTG (SEQ ID NO:16) and CAGCATTGCC GTCC T GGGTGAGAGTCTCTC (SEQ ID NO:17) were obtained from Sigma Biochemicals for generating the common form by site directed mutagenesis, utilizing Site Directed Mutagenesis kit (Stratagene) and the MMP-9 cDNA in the mammalian expression vector, pCl-Neo. The resulting MMP-9 cDNA containing the common allele (the common A allele encoding for 279Gln) was verified by restriction analysis.

An airway epithelial cell line, AALEB, was transfected with 2 μg each of an expression plasmid, pCl-neo, containing the 279Gln common variant of MMP-9 and separately the 279Arg rare variant. The culture media was harvested from untreated cells as a control and 48 hours after transfection with the control vector alone or the vector expressing the MMP-9 variants. The medium from each of these cultures (25 μL) was mixed with loading buffer and subjected to zymography. While the media from untreated control cells or cells transfected with control plasmid showed little MMP-9 and MMP-2 activity, dramatically increased MMP-9 activity was observed in lanes that contained media from cells transfected with the expression vectors for 279Gln common variant of MMP-9 and the 279Arg rare variant, as shown in FIG. 4. The dimer form of MMP-9 also showed activity at a higher molecular weight. However, the activity for MMP-2 in AALEB cells transfected with MMP-9 expression vectors was similar to that observed in controls. While non-transfected AALEB cells express low levels of MMP-9 and MMP-2, increased levels of MMP-9 were expressed in response to the transfection of MMP-9 expression vectors. Activity in both lanes 1 and 2 of FIG. 4 is similar, indicating that both the 279Gln common variant of MMP-9 and the 279Arg rare variant degrade gelatin, contained in the polyacrylamide gel as a substrate, with similar efficiency.

### EXAMPLE 10

### Expression of MMP-9 Gln279Arg Polymorphism in Bacterial Cells

The 279Gln common variant of MMP-9 and the 279Arg rare variant were expressed in a bacterial expression system, providing purified forms of the two MMP-9 variants. The cDNA constructs were subcloned into a bacterial expression plasmid containing a Thio-Histidine tag (Invitrogen, San Diego, Calif.). The cDNAs for 279Gln common variant of MMP-9 and the 279Arg rare variant were digested out of the pCl-neo expression plasmid with Eco RI and Not I and ligated into a multiple cloning site of the expression vector, pThioHis, expressed as fusions with a modified version of the E coli protein Thioredoxin (Invitrogen, Carlsbad, Calif.). The pThioHis B was used for this expression to have the sequence “in frame” with the histidine tag, permitting purification of the thioredoxin fusion on a metal-chelating resin. Isoproplyl-β-D-thiogalactopyranoside (ITPG) was used to induce expression of the protein.

After subcloning, the plasmids were transformed into Top10 Esherishia coli bacteria (Invitrogen, Carlsbad, Calif.) and the presence of the correct inserts verified. The pCl-neo plasmids containing the 279Gln common variant of MMP-9 and the 279Arg rare variant were sequenced in both directions, showing that all bases were identical to the MMP-9 sequence of SEQ ID NO:11 with the only base changed between the two cDNAs being the SNP, causing the Gln279Arg polymorphism.

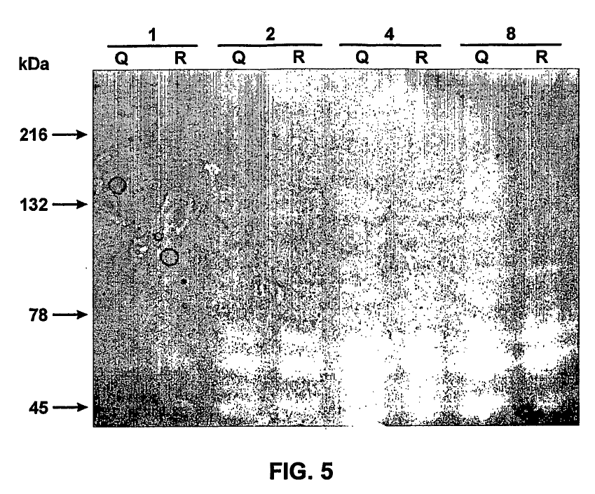

FIG. 5 shows a time course experiment using bacteria expressing the 279Gln common variant and the 279Arg rare variant, with protein synthesis induced with ITPG. Bacteria were lysed by ultra-sonication at time points 0, 1, 2, 4, 8, and 24 hours, following treatment with IPTG and bacterial proteins analyzed by zymography. While only faint bands for MMP-9 activity were detected at 1 and 2 hours after IPTG treatment, MMP-9 activity increased at 4 and 8 hours. Both lower molecular weight bands, indicative of cleavage products of the expressed MMP-9, and higher molecular weight bands, indicative of dimers, were detected.

### EXAMPLE 11

### Purified MMP-9 Preparations

Using the E. coli system of Example 10, purified preparations of the 279Gln common variant of MMP-9 and the 279Arg rare variant were made. Large quantities of transfected bacteria were grown using ITPG and the cells lysed by a series of freeze-thaw cycles combined with ultrasound sonification in binding buffer (50 mM NaPO₄, 0.5 mM NaCl and 0.333 mM imidazole). The lysate was filtered through a 0.8 μm pore filter, and the purified lysate incubated with nickel-containing ProBond Resin (Invitrogen) to bind the fusion proteins consisting of thioredoxin and the MMP-9 variants. After binding, the resin was washed several times with a wash buffer (50 mM NaPO₄ 0.5 mM NaCl and 22.333 mM imidazole) to remove all non-nickel-binding proteins in the lysate. The resin was then loaded onto a column with binding buffer and the proteins binding to the resin were eluted with elution buffer (50 mM NaPO₄, 0.5 mM NaCl and 250 mM imidazole) and collected into 0.5 mL fractions. Alternatively, the proteins were eluted with a low pH buffer or by competition with histidine. The resulting fractions containing the pro-MMP-9 proteins, which must be cleaved to show enzyme activity, were then activated, such as with a 1 mM concentration of aminophenylmercuric acetate (APMA) for one hour at 37° C., to cleave the thioredoxin with the pro-enzyme in the remaining protein being highly activated and showing a 92 kDa band in a zymogram.

### EXAMPLE 12

### Drug Discovery Using MMP-9 Gln279Arg Polymorphism Expression Systems

Either mammalian cell culture media containing the 279Gln common or 279Arg rare variant enzyme as in Example 9, or purified 279Gln common or 279Arg rare variant enzyme obtained using a bacterial expression system as in Examples 10 and 11 are obtained. The media or purified enzyme are used to screen for inhibitors of the 279Arg rare variant, and differentially to screen for inhibitors of the 279Arg rare variant which do not affect, or affect to a lesser degree, the 279Gln common variant. Specific inhibitors for the rare 279Arg variant are potential drugs to decrease the risk of developing COPD or to reduce the progression of this disease in the high-risk population.

To identify inhibitors that are specific for the rare 279Arg variant, the purified enzymes (MMP-9 279Gln and 279Arg) are incubated with a series of potential inhibitors of the MMP-9 enzyme. Because of structural differences between the two enzymes in the substrate binding region, it is hypothesized that the rare 279Arg variant can be inhibited by one or more chemical compounds while the 279Gln variant is not. Following incubation with various test chemical compounds, the enzyme activity toward gelatin is tested either by zymography or in a high throughput system by analyzing the digestion of gelatin in a 96-well reaction system followed by analyzing the color reaction by color absorbance in a microplate reader at 450 nm (Molecular Devices, Sunnyvale, Calif.).

The preceding examples can be repeated with similar success by substituting the generically or specifically described reactants and/or operating conditions of this invention for those used in the preceding examples.

Although the invention has been described in detail with particular reference to these preferred embodiments, other embodiments can achieve the same results. Variations and modifications of the present invention will be obvious to those skilled in the art and it is intended to cover all such modifications and equivalents. The entire disclosures of all references, applications, patents, and publications cited above are hereby incorporated by reference.

What is claimed is:

1. A method for determining the susceptibility of an individual to a chronic obstructive pulmonary disorder (COPD), comprising the step of determining the presence of an exon 6 codon 279 Gln/Arg single nucleotide polymorphism within the matrix metalloproteinase-9 (MMP-9) locus in a biological sample obtained from the individual, wherein the 279 arginine polymorphism indicates susceptibility to chronic obstructive pulmonary disorder.

2. The method of claim 1, further comprising use of an isolated nucleic acid molecule to detect the codon 279 Gln/Arg single nucleotide polymorphism.

3. The method of claim 2, wherein the isolated nucleic acid molecule is DNA, cDNA or mRNA.

4. The method of claim 2, wherein the isolated nucleic acid molecule is a single-stranded or double-stranded nucleic acid molecule.

5. The method of claim 2, wherein the isolated nucleic acid molecule is a probe which hybridizes under stringent conditions to a particular allele of the polymorphism.

6. The method of claim 5, wherein the probe comprises the sequence 5′-CTCTACACCCGGGACGGCAATG (SEQ ID NO:1).

7. The method of claim 5, wherein the probe comprises the sequence 5′-ACTCTACACCCAGGACGGCAATGC (SEQ ID NO:2).

8. The method of claim 2, further comprising use of a nucleotide primer which amplifies a particular allele of the polymorphism.

9. The method of claim 8, wherein the nucleotide primer comprises a 5′-TCTCCCCCTTTCCCACATC (SEQ ID NO:3) sense primer or a 5′-TGTGCTGTCTCCGCCTTCT (SEQ ID NO:4) antisense primer.

10. The method of claim 1, wherein determining the presence of an exon 6 codon 279 Gln/Arg single nucleotide polymorphism within the MMP-9 locus comprises testing expressed protein for the presence or absence of arginine in the 279 position.

11. A method of determining the efficacy of a substance to inhibit the 279Arg MMP-9 enzyme for use as a therapeutic or preventive agent for COPD, the method comprising the steps of:
providing the 279Arg MMP-9 enzyme; and
testing the substance for inhibition of the 279Arg MMP-9 enzyme.

12. The method of claim 11, wherein providing the 279Arg MMP-9 enzyme comprises inserting a gene expressing the 279Arg MMP-9 enzyme into a cell line.

13. The method of claim 12, wherein the gene expressing the 279Arg MMP-9 enzyme is SEQ ID NO:11 where 841 n is guanine (G).

14. The method of claim 11, further comprising the steps of:
providing the 279Gln MMP-9 enzyme;
testing the substance for inhibition of the 279Gln MMP-9 enzyme; and
comparing the results obtained for inhibition of the 279Arg MMP-9 enzyme with results obtained for inhibition of the 279Gln MMP-9 enzyme.

15. The method of claim 11, wherein the 279Arg MMP-9 enzyme is purified enzyme.

16. The method of claim 14, wherein the 279Arg MMP-9 enzyme and the 279Gln MMP-9 enzyme are each purified enzyme.

17. The method of claim 14, wherein the gene expressing the 279Gln MMP-9 enzyme is SEQ ID NO:11 where 841 n is adenine (A).

18. A method of determining the efficacy of a substance to inhibit a 279Arg MMP-9 enzyme without substantially inhibiting a 279Gln MMP-9 enzyme for use as a therapeutic or preventive agent for COPD, the method comprising the steps of:
providing the 279Arg MMP-9 enzyme;
testing the substance for inhibition of the 279Arg MMP-9 enzyme;
providing the 279Gln MMP-9 enzyme;
testing the substance for inhibition of the 279Gln MMP-9 enzyme;
comparing the results obtained for inhibition of the 279Arg MMP-9 enzyme with results obtained for inhibition of the 279Gln MMP-9 enzyme; and
selecting the substance which inhibits the 279Arg MMP-9 enzyme without substantially inhibiting the 279Gln MMP-9 enzyme.

19. The method of claim 18, wherein providing the 279Arg MMP-9 enzyme and the 279Gln MMP-9 enzyme comprises inserting a gene expressing the 279Arg MMP-9 enzyme into a first cell line and inserting a gene expressing the 279Gln MMP-9 enzyme into a second cell line.

20. The method of claim 19, wherein the gene expressing the 279Arg MMP-9 enzyme is SEQ ID NO:11 where 841 n is G and the gene expressing the 279Gln MMP-9 enzyme is SEQ ID NO:11 where 841 n is A.

21. The method of claim 18, wherein the 279Arg MMP-9 enzyme and the 279Gln MMP-9 enzyme are each purified enzyme.

22. A method of treating a patient with COPD or at risk for developing COPD, comprising the steps of:
determining the presence of an exon 6 codon 279 Gln/Arg single nucleotide polymorphism within the MMP-9 locus in a biological sample obtained from the patient;
administering an MMP-9 inhibitor to the patient with a 279 arginine polymorphism.

23. The method of claim 22, wherein the MMP-9 inhibitor is a selective 279Arg MMP-9 enzyme inhibitor.
